(19) United States
(12) Patent Application Publication
(10) Pub. No.: US 2017/0172872 A1
(43) Pub. Date: Jun. 22, 2017

(54) PROPELLANT-FREE DEODORANTS AND/OR ANTIPERSPIRANTS HAVING SPECIFIC PRESERVATIVE-AGENT COMBINATIONS

(71) Applicant: Henkel AG & Co. KGaA, Duesseldorf (DE)

(72) Inventors: Bernhard Banowski, Duesseldorf (DE); Rainer Simmering, Grevenbroich (DE); Thomas Doering, Dormagen (DE); Melanie Rauschenberg, Kamen (DE)

(73) Assignee: Henkel AG & Co. KGaA, Duesseldorf (DE)

(21) Appl. No.: 15/377,665

(22) Filed: Dec. 13, 2016

(30) Foreign Application Priority Data
Dec. 17, 2015 (DE) 10 2015 225 694.4

(51) Int. Cl. A61K 8/26; A61K 8/23; A61K 8/49; A61K 8/42; A61K 8/36; A61K 8/44; A61K 8/41; A61Q 15/00; A61K 8/34

(52) U.S. Cl. CPC A61K 8/26; A61Q 15/00; A61K 8/23; A61K 8/4953; A61K 8/34; A61K 8/361; A61K 8/4926; A61K 8/44; A61K 8/36; A61K 8/416; A61K 8/347; A61K 8/42; A61K 2800/524; A61K 2800/5922

(57) ABSTRACT

The present invention relates to propellant-containing deodorant and/or antiperspirant compositions that include specific preservative-agent combinations. The present invention also relates to a method for preventing and/or reducing the perspiration of the body and/or for preventing and/or reducing body odor by using the cosmetic agents according to the invention and the use of a certain preservative-agent combination to preserve propellant-containing deodorant and/or antiperspirant compositions.

### FIELD OF THE INVENTION

The present invention generally relates to propellant-containing deodorant and/or antiperspirant compositions that include at least one specific preservative-agent combination in addition to at least one deodorant active substance and/or at least one antiperspirant aluminum salt. The previously mentioned compositions have excellent deodorant and/or antiperspirant performance together with good preservation.

Furthermore, the present invention relates to a non-therapeutic cosmetic method for preventing and/or reducing body odor, wherein a cosmetic agent according to the invention is applied to the skin, particularly to the skin of the armpits, and remains on the skin for at least 1 hour, preferably for at least 2 hours, more preferably for at least 4 hours, particularly for at least 6 hours.

Finally, the present invention relates to the use of specific preservative-agent combinations to preserve propellant-containing deodorant and/or antiperspirant compositions.

### BACKGROUND OF THE INVENTION

Because of the composition of cosmetic agents, cosmetic agents can serve as a nutrient medium for microorganisms, such as bacteria, yeasts, and fungi. These microorganisms can make cosmetic products unusable, for example because of the formation of mold. Microbial contamination of the consumer can also occur. Furthermore, these microorganisms can change ingredients of the cosmetics and thus form substances having undesired effects such as sensitization or skin irritation. In order to prevent these undesired consequences and ensure a certain minimum shelf life of the cosmetics, the cosmetics must be preserved. Because preservative agents themselves have the potential to cause irritation, the use of preservative agents in cosmetics is strictly controlled and is regulated by law.

The skin microbiota has a crucial influence on different cosmetic parameters. Pathogenic microbes such as Staphylococcus aureus play a crucial role in the production of skin impurities. The latest studies also indicate that an unbalanced skin microbiota can have an influence on skin aging, because undesired microbes lead to increased immune defense of the skin, which in turn leads to increased inflammatory responses, in the course of which skin aging markers are stimulated.

Therefore, there is still a need for preservative-agent compositions that prevent the colonization of the product by microbes undesired for the skin and that do not impair the natural skin flora or impair it only slightly.

The mixing of various antimicrobial substances to increase the antimicrobial activity is known in general. WO 03/043593 A1 proposes combining conventional antibacterial substances, such as triclosan, phenoxyethanol, or hexetidine, with ethyl lauroyl arginate to strengthen the antibacterial effect. WO 2007/014580 A1 proposes preservative-agent mixtures which, in addition to ethyl lauroyl arginate, include salts of organic or inorganic acids, particularly sodium citrate, sodium acetate, sodium glutamate, sodium fumarate, sodium malate, sodium gluconate, sodium laurate, sodium lactate, sodium hexametaphosphate, sodium tert-butylhydroquinate, sodium propylparabenate, or the hydrochlorides of glucosamine or ethanolamine. EP 1414394 B1 discloses cosmetic compositions that include a preservative-agent mixture consisting of ethyl lauroyl arginate and parabens, imidazolyl urea, phenoxyethanol, DMDM hydantoin, 2-methyl-5-chloro-3,4-isothiazolinone/2-methyl-3,4-isothiazolinone, and Quaternium-15.

Therefore, there is still a need to provide antimicrobial compositions that are highly effective in a low usage amount.

Therefore, the problem addressed by the present invention is that of providing propellant-containing deodorant and/or antiperspirant compositions that have excellent deodorant and/or antiperspirant performance together with good preservation. In particular, synergistic preservative-agent mixtures that are highly effective at low concentrations and that enable the production of low-irritation and low-sensitization deodorant and/or antiperspirant compositions because of usage amounts that are reduced in total should be found. Furthermore, no preservative agents that are felt to be hazardous by the consumer should be used. In addition, the deodorant and/or antiperspirant performance and the storage stability of these agents should not be impaired.

It has now surprisingly been found that the use of certain mixtures of preservative agents in propellant-containing deodorant and/or antiperspirant compositions leads to a synergistic effect with regard to the preservative effect. Therefore, the amount of preservative agents used can be reduced without negatively affecting the preservative effect. Because of the reduced amount of preservative agents, the deodorant and/or antiperspirant compositions according to the invention are low-irritation and low-sensitization compositions. Furthermore, by using certain preservative-agent mixtures, excellent preservation can be achieved even without using preservative agents that are felt to be hazardous by the consumer, such as parabens. Furthermore, the addition of the preservative-agent mixtures does not lead to a negative effect on the deodorant and/or antiperspirant performance.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent containing, in a cosmetically acceptable carrier, 0.0005 to 35 wt %, with respect to the total weight of the propellant-free cosmetic agent, of at least one active substance, selected from the group of deodorant active substances, antiperspirant aluminum salts, and mixtures thereof; 2.0 to 95 wt %, with respect to the total weight of the propellant-containing cosmetic agent, of at least one propellant; and at least two preservative agents, selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

The use of at least two preservative agents, selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine, to preserve propellant-containing deodorant and/or antiperspirant compositions.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates to a cosmetic agent containing, in a cosmetically acceptable carrier,
a) 0.0005 to 35 wt %, with respect to the total weight of the propellant-free cosmetic agent, of at least one active substance, selected from the group of deodorant active substances, antiperspirant aluminum salts, and mixtures thereof,
b) 2.0 to 95 wt %, with respect to the total weight of the propellant-containing cosmetic agent, of at least one propellant, and
c) at least two preservative agents, selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

According to the invention, the term “deodorant active substance” is understood to mean an active substance that reduces and/or prevents the creation of body odor caused by the bacterial decomposition of sweat, particularly armpit sweat. However, the preservative agents specified under feature c) above and ethanol, which is possibly used as a constituent of the cosmetic carrier, do not fall thereunder.

In this document, the specification of wt % relates to the total weight of the propellant-free cosmetic agents, unless otherwise indicated.

The cosmetic agent includes the constituents a) to c) in a cosmetically acceptable carrier. Said carrier preferably includes at least one component, selected from water, a C₁-C₄ alcohol, a cosmetic oil that is liquid under normal conditions, and mixtures thereof. The cosmetic oils that are liquid under normal conditions are immiscible with water and are neither odorous substances nor ethereal oils. In the sense of the present invention, “normal conditions” are a temperature of 20° C. and a pressure of 1,013 hPa.

Suitable cosmetic carriers are, for example, aqueous or aqueous-alcoholic carriers. The term “aqueous or aqueous-alcoholic carriers” is understood to mean carriers that include more than 5.0 wt % of water or more than 5 wt % of water and at least one C₁-C₄ alcohol, with respect to the total weight of the propellant-free cosmetic agent. Aqueous carriers preferably include free water in a total amount of 10 to 96 wt %, preferably 15 to 80 wt %, preferably 30 to 70 wt %, particularly 40 to 60 wt %, with respect to the total weight of the propellant-free cosmetic agent. In aqueous-alcoholic carriers, ethanol is preferably contained in a total amount of 0.1 to 60 wt %, preferably 0.5 to 30 wt %, preferably 1.0 to 10 wt %, particularly 1.0 to 9.0 wt %, with respect to the total weight of the propellant-free cosmetic agent.

The previously mentioned aqueous and aqueous-alcoholic carriers can additionally include at least one (C₂-C₆) alkyl alcohol having 2 or 3 hydroxy groups, particularly 1,2-propylene glycol, 1,3-propylene glycol, glycerol, and 1,3-butylene glycol.

Furthermore, it is especially preferred in this context that liquid cosmetic oils are used as cosmetic carriers. These cosmetic oils can be selected from the group of (i) volatile non-silicone oils, particularly liquid paraffin oils and isoparaffin oils, such as isodecane, isoundecane, isododecane, isotridecane, isotetradecane, isopentadecane, isohexadecane, and isoeicosane; (ii) non-volatile non-silicone oils, particularly the esters of linear or branched saturated or unsaturated C₂₋₃₀ fatty alcohols with linear or branched saturated or unsaturated C₂₋₃₀ fatty acids, which can be hydroxylated, the C₈-C₂₂ fatty alcohol esters of monovalent or polyvalent C₂-C₇ hydroxycarboxylic acids, the triethyl citrates, the dicarboxylic acid esters of linear or branched C₂-C₁₀ alkanols, the products of the addition of ethylene oxide and/or propylene oxide to mono- or polyhydric C₃₋₂₂ alkanols, which can optionally be esterified, the symmetrical, unsymmetrical, or cyclic esters of carbonic acid with fatty alcohols, the esters of dimers of unsaturated C₁₂₋₂₂ fatty acids with monohydric, linear, branched, and cyclic C₂₋₁₈ alkanols or C₂₋₆ alkanols, the benzoic acid esters of linear or branched C₈₋₂₂ alkanols, such as benzoic acid C₁₂₋₁₅ alkyl esters and benzoic acid isostearyl ester and benzoic acid octyldodecyl ester, the synthetic hydrocarbons, such as polyisobutylene and polydecenes, the alicyclic hydrocarbons; and (iii) mixtures thereof.

According to the invention, the term “volatile cosmetic oil” refers to cosmetic oils that have a vapor pressure of 2.66 Pa to 40,000 Pa (0.02 to 300 mm Hg), preferably 10 to 12,000 Pa (0.1 to 90 mm Hg), more preferably 13 to 3,000 Pa (0.1 to 23 mm Hg), particularly 15 to 500 Pa (0.1 to 4 mm Hg), at 20° C. and an ambient pressure of 1,013 hPa. Furthermore, in the sense of the present invention, the term “non-volatile cosmetic oils” is understood to mean cosmetic oils that have a vapor pressure of less than 2.66 Pa (0.02 mm Hg) at 20° C. and an ambient pressure of 1,013 hPa.

Also preferred according to the invention is the use of mixtures of the aforementioned cosmetic oils, particularly of non-volatile and volatile cosmetic oils, because in this way parameters such as skin feel, visibility of the residue, and stability of the cosmetic agent according to the invention can be set and the agent thus can be better adapted to the needs of the consumers.

In the context of the present invention, it is preferred if the cosmetic oil that is liquid at 20° C. and 1,013 hPa is used in a total amount of 1.0 to 75 wt %, preferably 2.0 to 95 wt %, preferably 5.0 to 70 wt %, more preferably 10 to 60 wt %, particularly 15 to 50 wt %, with respect to the total weight of the propellant-free cosmetic agent.

Furthermore, preferred cosmetic carriers are O/W emulsions, W/O emulsions, and multiple emulsions, particularly W/O/W and O/W/O emulsions.

The cosmetic agent according to the invention includes at least one deodorant active substance and/or at least one antiperspirant aluminum salt as the first essential constituent a).

Preferred cosmetic agents include certain deodorant active substances. Therefore, in the context of the present invention, it is advantageous if the cosmetic agent includes at least one deodorant active substance, selected from the group of (i) silver salts; (ii) alkane diols having 5 to 12 carbon atoms, particularly 3-(2-ethylhexyloxy)-1,2-propanediol, 1,2-hexanediol, and 1,2-octanediol; (iii) triethyl citrates; (iv) active substances against exo-esterases, particularly against aryl sulfatase, lipase, beta-glucuronidase, and cystathionine β-lyase; (v) cationic phospholipids; (vi) odor absorbers, particularly silicates, such as montmorillonite, kaolinite, illite, beidellite, nontronite, saponite, hectorite, bentonite, smectite and talc, zeolites, zinc ricinoleate, cyclodextrins; (vii) deodorizing ion exchangers; (viii) microbe-inhibiting agents; (ix) prebiotic components; and (x) mixtures thereof.

Preferred silver salts and/or silver complexes are, for example, silver lactate, silver nitrate, silver acetate, silver sulfate, and silver citrate and combinations thereof with phyllosilicates and/or talc. Preferably, 1,2-pentanediol, 1,2-hexanediol, 1,2-octanediol, 1,2-decanediol, and mixtures thereof are used as an alkane diol having 5 to 12 carbon atoms. Especially preferred is a mixture of 1,2-hexanediol and 1,2-octanediol at a weight ratio of 5:1 to 1:5. It can also be preferred that a combination of at least one alkane diol with tropolone is used.

Silicates serve as odor absorbers which, at the same time, can also advantageously support the rheological properties of the cosmetic agents according to the invention. The silicates that are especially advantageous according to the invention include, above all, phyllosilicates, particularly montmorillonite, kaolinite, illite, beidellite, nontronite, saponite, hectorite, bentonite, smectite and talc. Other advantageous odor absorbers are, for example, zeolites, zinc ricinoleate, cyclodextrins, certain metal oxides, such as aluminum oxide, and chlorophyll.

Furthermore, the deodorant active substance can be selected from the group of terpene alcohols such as farnesol, chlorophyllin-copper complexes, α-monoalkylglycerol ether having a branched or linear saturated or unsaturated, possibly hydroxylated C₆-C₂₂ alkyl residue, especially preferably α-(2-ethylhexyl)glycerol ether, commercially available as Sensiva® SC 50 (from Schülke & Mayr), carboxylic acid esters, particularly carboxylic acid monoesters of mono-, di-, and trigylcerol (particularly glycerol monolaurate, diglycerol monocaprate, diglycerol monolaurate, triglycerol monolaurate, and triglycerol monomyristate), and plant extracts (for example, green tea and constituents of linden blossom oil).

Other preferred deodorant active substances are selected from prebiotic components, which, according to the invention, should be understood to mean components that inhibit only or at least predominantly the odor-forming microbes of the skin microbiota but not the desired microbes, i.e., the microbes that do not form odors.

Other preferred deodorant active substances are selected from the microbe-inhibiting perfume oils and the Deosafe perfume oils, which are available from the company Symrise, formerly Haarmann and Reimer.

The enzyme inhibitors include substances that inhibit the enzymes responsible for the decomposition of sweat, particularly arylsulfatase, β-glucuronidase, aminoacylase, the ester-cleaving lipases, and the lipoxigenase. Examples of such substances are trialkyl citric acid esters, particularly triethyl citrate, or zinc glycinate.

The deodorant active substances can be used individually and in mixtures. Especially preferred are α-(2-ethylhexyl)glycerol ether, diglycerol monocaprate, 2-methyl-4-phenylbutan-2-ol, mixtures of phenoxyethanol and α-(2-ethylhexyl)glycerol ether, and mixtures of aryl alcohols, particularly phenoxyethanol, with α-(2-ethylhexyl)glycerol ether and diglycerol monocaprate.

The at least one deodorant active substance is advantageously used in the cosmetic agents according to the invention in certain amount ranges. Therefore, preferred cosmetic agents of the present invention are characterized in that said cosmetic agents include—with respect to the total weight of the propellant-free cosmetic agents—0.0005 to 20 wt %, preferably 0.1 to 12 wt %, preferably 0.1 to 10 wt %, particularly 0.1 to 2.0 wt %, of at least one deodorant active substance. If a mixture of deodorant active substances is used, the previously stated amount specifications relate to the mixture of said active substances. The use of the previously stated amounts ensures sufficient deodorant performance. Furthermore, these amounts do not have a negative influence on the preservative effect of the mixture of at least two preservative agents.

In addition to or in combination with the at least one deodorant active substance, the cosmetic agents according to the invention can additionally include at least one antiperspirant aluminum salt. Therefore, in the context of the present invention, it is preferred if the cosmetic agent includes at least one antiperspirant aluminum salt, selected from the group of (i) water-soluble astringent inorganic salts of aluminum, particularly aluminum chlorohydrate, aluminum sesquichlorohydrate, aluminum dichlorohydrate, aluminum hydroxide, potassium aluminum sulfate, aluminum bromohydrate, aluminum chloride, aluminum sulfate; (ii) water-soluble astringent organic salts of aluminum, particularly aluminum chlorohydrex propylene glycol, aluminum chlorohydrex polyethylene glycol, aluminum propylene glycol complexes, aluminum sesquichlorohydrex propylene glycol, aluminum sesquichlorohydrex polyethylene glycol, aluminum dichlorohydrex propylene glycol, aluminum dichlorohydrex polyethylene glycol, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate; and (iii) mixtures thereof.

Especially preferred inorganic aluminum salts are selected from aluminum chlorohydrate, particularly aluminum chlorohydrate having the general formula [Al₂(OH)₅Cl.1-6 H₂O]ₙ, preferably [Al₂(OH)₅Cl.2-3 H₂O]ₙ, which can be present in unactivated (polymerized) or activated (depolymerized) form, and aluminum chlorohydrate having the general formula [Al₂(OH)₄Cl₂.1-6 H₂O]ₙ, preferably [Al₂(OH)₄Cl₂.2-3 H₂O]ₙ, which can be present in unactivated (polymerized) or activated (depolymerized) form.

Antiperspirant aluminum salts especially preferred according to the invention are selected from “activated” aluminum salts, which are also called antiperspirant active substances “having enhanced activity.” Such active substances are known in the prior art and are also commercially available. Activated aluminum salts are generally produced by thermally treating a diluted solution of the corresponding salt (e.g., a solution having 10 wt % of salt) in order to increase the HPLC peak-4-to-peak-3 area ratio of said salt. The activated salt can then be dried, particularly spray-dried, into a powder. Besides spray drying, drum drying is also suitable, for example. Activated aluminum salts typically have an HPLC peak-4-to-peak-3 area ratio of at least 0.4, preferably at least 0.7, particularly at least 0.9, wherein at least 70% of the aluminum can be associated with these HPLC peaks.

According to the invention, it is also possible to use antiperspirant aluminum salts as non-aqueous solutions or solubilizates of an activated antiperspirant aluminum salt, for example according to document U.S. Pat. No. 6,010,688 A. Such aluminum salts are stabilized against the loss of the activation of the salt by the addition of an effective amount of a polyhydric alcohol that has 3 to 6 carbon atoms and 3 to 6 hydroxyl groups, preferably propylene glycol, sorbitol, and pentaerythritol.

Also especially preferred are complexes of activated antiperspirant aluminum salts with a polyhydric alcohol that include 20 to 50 wt %, preferably 20 to 42 wt %, of activated antiperspirant aluminum salt and 2 to 16 wt % of molecularly bound water, wherein the remainder consists of 100 wt % of at least one polyhydric alcohol having 3 to 6 carbon atoms and 3 to 6 hydroxyl groups. Propylene glycol, propylene glycol/sorbitol mixtures, and propylene glycol/pentaerythritol mixtures are preferred such alcohols.

In the context of the present invention, it is also possible to use basic calcium-aluminum salts, which are disclosed, for example, in document U.S. Pat. No. 2,571,030, as antiperspirant aluminum salts. Said salts can be obtained by reacting calcium carbonate with aluminum chlorohydroxide or aluminum chloride and aluminum powder or by adding calcium chloride dihydrate to aluminum chlorohydroxide. However, it is also possible to use aluminum-zirconium complexes that are buffered with salts of amino acids, particularly with alkali and alkaline-earth glycinates.

Also usable as antiperspirant activated aluminum salts preferred according to the invention are the aluminum salts which are preferably stabilized by means of amino acids, particularly glycine, hydroxyalkanoic acids, particularly glycolic acid and lactic acid, or betaines.

Other preferred activated aluminum salts are those of the general formula Al₂(OH)₆₋ₐXa, wherein X represents Cl, Br, I, or NO₃ and “a” represents a number from 0.3 to 5, preferably 0.8 to 2.5, particularly 1 to 2, and therefore the molar ratio of Al:X is 0.9:1 to 2.1:1. Such activated antiperspirant aluminum salts are disclosed, for example, in document U.S. Pat. No. 6,074,632 A. Especially preferred is aluminum chlorohydrate (i.e., X represents Cl in the formula stated above) and specifically 5/6 basic aluminum chlorohydrate with “a”=1, the molar ratio of aluminum to chlorine thus being 1.9:1 to 2.1:1.

Antiperspirant aluminum salts especially preferred according to the invention have a molar metal-to-chloride ratio of 1.9 to 2.1. The metal-to-chloride ratio of aluminum sesquichlorohydrates likewise especially preferred in the context of the invention is 1.5:1 to 1.8:1.

The at least one antiperspirant aluminum salt is advantageously used in the cosmetic agents according to the invention in certain amount ranges. Therefore, preferred cosmetic agents of the present invention are characterized in that said cosmetic agents include—with respect to the total weight of the propellant-free cosmetic agents—0.1 to 35 wt %, preferably 0.5 to 25 wt %, preferably 1 to 15 wt %, more preferably 1.5 to 10 wt %, particularly 2.0 to 8.0 wt %, of at least one antiperspirant aluminum salt. If a mixture of antiperspirant aluminum salts is used, the previously stated amount specifications relate to the mixture of said salts. The use of the previously stated amounts ensures sufficient deodorant and antiperspirant performance. By means of the previously stated specific preservative-agent combination, excellent preservative performance can be ensured even if the previously stated amounts of the antiperspirant aluminum salt are used.

The cosmetic agents according to the invention include at least one propellant as the second essential constituent b). Therefore, cosmetic agents according to the invention are formulated as a spray application. The spray application occurs by means of a spraying device, which includes a filling of the antiperspirant cosmetic agent according to the invention in a container, which cosmetic agent is liquid, viscously flowable, in suspension form, or powdery. Said spraying device is under the pressure of a propellant (compressed-gas cans, compressed-gas dispensers, aerosol dispensers). The containers have a removal device, preferably in the form of valves, which make it possible to remove the contents as a mist, fume, foam, powder, paste, or liquid jet. Above all, cylindrical vessels composed of metal (aluminum, tinplate, volume capacity preferably at most 1,000 ml), protected or non-splintering glass or plastic (volume capacity preferably at most 220 ml), or splintering glass or plastic (volume capacity preferably 50 to 400 ml) are considered as containers for the spraying devices. The packaging for the agents according to the invention can be opaque, transparent, or translucent.

Propellants (propellant gases) suitable in the context of the invention are, for example, propane, propene, n-butane, isobutane, isobutylene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, individually and in mixtures thereof. Hydrophilic propellant gases, such as carbon dioxide, can also be advantageously used according to the present invention if the proportion of hydrophilic gases is low and lipophilic propellant gas (e.g., propane/butane) is present in excess. Therefore, preferred cosmetic agents according to the invention are characterized in that said cosmetic agents include at least one propellant, selected from the group of propane, propene, n-butane, isobutane, isobutylene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, carbon dioxide, and mixtures thereof.

According to the invention, the at least one propellant is preferably used in certain total amounts. Therefore, preferred cosmetic agents of the present invention are characterized in that said cosmetic agents include—with respect to the total weight of the propellant-containing cosmetic agent—2.0 to 90 wt %, preferably 30 to 85 wt %, preferably 35 to 80 wt %, particularly 40 to 75 wt %, of at least one propellant. If a mixture of propellants is used, the previously stated amount specifications relate to the mixture of said propellants. The use of the previously stated amounts guarantees sufficient sprayability and ensures complete emptying of the aerosol can. Because of the even application of the cosmetic agent by using the previously stated propellants and propellant amounts, excellent deodorant and/or antiperspirant performance is ensured.

The cosmetic agents according to the invention include at least two preservative agents, selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine, as the third essential constituent c). These compounds have the following chemical names and CAS numbers:

| | | CAS |
|---|---|---|
| Preservative agent | Chemical name | number |
| Hexetidine | 1,3-Bis(2-ethylhexyl)hexahydro- | 141-94-6 |
| | 5-methyl-5-pyrimidiamine | |
| Benzyl alcohol | Benzyl alcohol | 100-51-6 |
| Undecylenic acid | Undec-10-enoic acid | 112-38-9 |
| Phenoxyisopropanol | 1-Phenoxypropan-2-ol | 770-35-4 |
| Piroctone olamine | 1-Hydroxy-4-methyl-6- | 68890-66-4 |
| | (2,4,4-trimethylpentyl)-2-pyridone | |
| Ethyl lauroyl | Ethyl-N-alpha-dodecanoyl-L-arginate | 60372-77-2 |
| arginate HCl | hydrochloride | |
| o-Cymen-5-ol | 4-Isopropyl-m-cresol | 3228-02-2 |
| Hexamidine | 4,4′-[1,6-hexanediylbis(oxy)] | 3811-75-4 |
| | bis[benzenecarboxamidine] | |
| Hexamidine | 2-Hydroxyethanesulfonic acid, compd. | 659-40-5 |
| diisethionate | with 4,4′-[hexane-1,6-diylbis(oxy)] | |
| | bis[benzenecarboxamidine] (2:1) | |
| Hexamidine paraben | p-Hydroxybenzoic acid, compd. with | 93841-83-9 |
| | 4,4′-[hexane-1,6-diylbis(oxy)]bis | |
| | [benzenecarboxamidine] (2:1) | |

These preservative-agent mixtures have a synergistic effect with respect to the antimicrobial activity and therefore lead to especially effective preservation of the cosmetic agents according to the invention. Furthermore, because of the synergistic effect, the usage amount of said preservative-agent mixtures can be reduced so that low-irritation and low-sensitization cosmetic agents are obtained. In addition, the deodorant and/or antiperspirant performance is not negatively affected by the use of the preservative-agent mixture.

The preservative effect can be increased further if the cosmetic agents include more than two of the previously mentioned preservative agents. Therefore, preferred cosmetic agents are characterized in that said cosmetic agents include at least three preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

Other preferred cosmetic agents according to the invention are characterized in that said cosmetic agents include at least four preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

Furthermore, according to the invention, cosmetic agents that additionally include at least five preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine, are advantageous.

In addition, in the context of the present invention, cosmetic agents that include at least six preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine, are preferred.

Finally, preferred are cosmetic agents according to the invention that include a mixture of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine as a preservative agent (c).

The cosmetic agents according to the invention can include at least one quaternary ammonium compound as a preservative agent. In the context of the present invention, it has proven to be advantageous if certain quaternary ammonium compounds are used. Therefore, in the context of the present invention, it is preferred if the cosmetic agent includes at least one compound, selected from the group of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and mixtures thereof, as quaternary ammonium compounds. The previously mentioned compounds are C₁₂-C₂₂-alkyltrimethylammonium bromides and chlorides, wherein the particular name represents the length of the alkyl chain of the particular compound. Therefore, behentrimonium chloride is C₂₂-alkyltrimethylammonium chloride, cetrimonium chloride means C₁₆-alkyltrimethylammonium chloride, laurtrimonium bromide represents C₁₂-alkyltrimethylammonium bromide, and steartrimonium bromide means C₁₈-alkyltrimethylammonium bromide. The use of such quaternary ammonium compounds, in combination with at least one other claimed preservative agent, has proven especially advantageous with respect to the synergistic increase in the preservative-agent performance of this mixture.

The cosmetic agents according to the invention preferably include the preservative-agent mixture in certain amount ranges. Therefore, preferred cosmetic agents according to the invention are characterized in that said cosmetic agents include—with respect to the total weight of the propellant-free cosmetic agent—0.001 to 10 wt %, preferably 0.005 to 7.0 wt %, preferably 0.01 to 4.0 wt %, particularly 0.05 to 2.0 wt %, of at least two preservative agents (c). The previously indicated amounts relate to the mixture of the preservative agents used. The use of such amounts of the preservative-agent mixture leads to excellent preservation of the cosmetic agents according to the invention. Furthermore, because of the synergistic effect of the preservative-agent mixture, the usage amount of the preservative-agent mixture can be reduced without negatively affecting the preservative performance. Therefore, the cosmetic agents according to the invention are especially low-irritation and low-sensitization cosmetic agents.

Especially preferred embodiments AF1 to AF24 of the cosmetic agents according to the invention are indicated in the table below (all specifications in wt %, with respect to the propellant-free cosmetic agent). Here, KM represents a mixture of at least two of the previously mentioned preservative agents.

| | AF1 | AF2 | AF3 | AF4 |
|---|---|---|---|---|
| Deodorant active | 0.0005-20 | 0.1-12 | 0.1-10 | 0.1-2.0 |
| substance | | | | |
| KM | 0.001-10 | 0.005-7.0 | 0.01-4.0 | 0.05-2.0 |
| Carrier ¹⁾ | Ad 100 | Ad 100 | Ad 100 | Ad 100 |
| | AF5 | AF6 | AF7 | AF8 |
| Antiperspirant | 0.1-35 | 0.5-25 | 1-15 | 2.0-8.0 |
| aluminum salt | | | | |
| KM | 0.001-10 | 0.005-7.0 | 0.01-4.0 | 0.05-2.0 |
| Carrier ¹⁾ | Ad 100 | Ad 100 | Ad 100 | Ad 100 |
| | AF9 | AF10 | AF11 | AF12 |
| Deodorant active | 0.0005-20 | 0.1-12 | 0.1-10 | 0.1-2.0 |
| substance | | | | |
| Antiperspirant | 0.1-35 | 0.5-25 | 1-15 | 2.0-8.0 |
| aluminum salt | | | | |
| KM | 0.001-10 | 0.005-7.0 | 0.01-4.0 | 0.05-2.0 |
| Carrier ¹⁾ | Ad 100 | Ad 100 | Ad 100 | Ad 100 |
| | AF13 | AF14 | AF15 | AF16 |
| Deodorant active | 0.0005-20 | 0.1-12 | 0.1-10 | 0.1-2.0 |
| substance ²⁾ | | | | |
| KM | 0.001-10 | 0.005-7.0 | 0.01-4.0 | 0.05-2.0 |
| Carrier ¹⁾ | Ad 100 | Ad 100 | Ad 100 | Ad 100 |
| | AF17 | AF18 | AF19 | AF20 |
| Antiperspirant | 0.1-35 | 0.5-25 | 1-15 | 2.0-8.0 |
| aluminum salt ³⁾ | | | | |
| KM | 0.001-10 | 0.005-7.0 | 0.01-4.0 | 0.05-2.0 |
| Carrier ¹⁾ | Ad 100 | Ad 100 | Ad 100 | Ad 100 |
| | AF21 | AF22 | AF23 | AF24 |
| Deodorant active | 0.0005-20 | 0.1-12 | 0.1-10 | 0.1-2.0 |
| substance ²⁾ | | | | |
| Antiperspirant | 0.1-35 | 0.5-25 | 1-15 | 2.0-8.0 |
| aluminum salt ³⁾ | | | | |
| KM | 0.001-10 | 0.005-7.0 | 0.01-4.0 | 0.05-2.0 |
| Carrier ¹⁾ | Ad 100 | Ad 100 | Ad 100 | Ad 100 |

¹⁾ aqueous, aqueous-alcoholic, or water-free carrier
²⁾ selected from mixture, comprising 1,2-hexanediol and 1,2-octanediol,
³⁾ aluminum chlorohydrate

The embodiments AF1 to AF24 are mixed with propellant (air, nitrous oxide, nitrogen, or CO₂) at a weight ratio of 20:80 or 15:85 or 40:60 or 10:90 or 5:95 or 90:10 or 98:2.

The preservative mixtures KM1 to KM1762 of the table below are used as the preservative mixture KM in each embodiment AF1 to AF24 above. Here, s represents sulfite, selected from the group of sodium sulfite, ammonium bisulfite, ammonium sulfite, potassium sulfite, potassium hydrogen sulfite, sodium bisulfite, sodium metabisulfite, and potassium metabisulfite and mixtures thereof; h represents hexetidine; b represents benzyl alcohol; u represents undecylenic acid; p represents phenoxyisopropanol; pi represents piroctone olamine; e represents ethyl lauroyl arginate HCl; a represents formic acid; bz represents benzethonium chloride; be represents behentrimonium chloride; cb represents cetrimonium bromide; cc represents cetrimonium chloride; lb represents laurtrimonium bromide; lc represents laurtrimonium chloride; sb represents steartrimonium bromide; sc represents steartrimonium chloride; aq represents a quaternary ammonium compound; oc represents o-cymen-5-ol; hx represents hexamidine; hd represents hexamidine diisethionate; hp represents hexamidine paraben. In the table, the specification h+b, for example, thus represents a mixture of hexetidine and benzyl alcohol. h+b+u accordingly represents the combination of hexetidine, benzyl alcohol, and undecylenic acid.

| KM1 | KM2 | KM3 | KM4 | KM5 | KM5 | KM6 | KM7 |
|---|---|---|---|---|---|---|---|
| s + h | h + b | b + u | u + p | p + pi | pi + e | e + a | a + bc |
| KM8 | KM9 | KM10 | KM11 | KM12 | KM13 | KM14 | KM15 |
| s + b | h + u | b + p | u + pi | p + e | pi + a | e + bc | a + cb |
| KM16 | KM17 | KM18 | KM19 | KM20 | KM21 | KM22 | KM23 |
| s + u | h + p | b + pi | u + e | p + a | pi + bc | e + cb | a + cc |
| KM24 | KM25 | KM26 | KM27 | KM28 | KM29 | KM30 | KM31 |
| s + p | h + pi | b + e | u + a | p + bc | pi + cb | e + cc | a + lb |
| KM32 | KM33 | KM34 | KM35 | KM36 | KM37 | KM38 | KM39 |
| s + pi | h + e | b + a | u + bc | p + cb | pi + cc | e + lb | a + lc |
| KM40 | KM41 | KM42 | KM43 | KM44 | KM45 | KM46 | KM47 |
| s + e | h + a | b + bc | u + cb | p + cc | pi + lb | e + lc | a + sb |
| KM48 | KM49 | KM50 | KM51 | KM52 | KM53 | KM54 | KM55 |
| s + a | h + bc | b + cb | u + cc | p + lb | pi + lc | e + sb | a + sc |
| KM56 | KM57 | KM58 | KM59 | KM60 | KM61 | KM62 | KM63 |
| s + bc | h + cb | b + cc | u + lb | p + lc | pi + sb | e + sc | a + bc |
| KM64 | KM65 | KM66 | KM67 | KM68 | KM69 | KM70 | KM71 |
| s + cb | h + cc | b + lb | u + lc | p + sb | pi + sc | e + bc | a + bz |
| KM72 | KM73 | KM74 | KM75 | KM76 | KM78 | KM79 | KM80 |
| s + cc | h + lb | b + lc | u + sb | p + sc | pi + bc | e + bz | s + lb |
| KM81 | KM82 | KM83 | KM84 | KM85 | KM86 | KM87 | KM88 |
| h + lc | b + sb | u + sc | p + bc | pi + bz | s + lc | h + sb | b + sc |
| KM89 | KM90 | KM91 | KM92 | KM93 | KM94 | KM95 | KM96 |
| u + bc | p + bz | s + sb | h + sc | b + bc | u + bz | s + sc | h + bc |
| KM97 | KM98 | KM99 | KM100 | KM101 | KM102 | KM103 | KM104 |
| b + bz | s + bc | h + bz | s + bz | bc + cb | cb + cc | cc + lb | lb + lc |
| KM105 | KM106 | KM107 | KM108 | KM109 | KM110 | KM111 | KM112 |
| lc + sb | sb + sc | sc + bc | bc + bz | bc + cc | cb + lb | cc + lc | lb + sb |
| KM113 | KM114 | KM115 | KM116 | KM117 | KM118 | KM119 | KM120 |
| lc + sc | sb + bc | sc + bz | bc + lb | cb + lc | cc + sb | lb + sc | lc + bc |
| KM121 | KM122 | KM123 | KM124 | KM125 | KM126 | KM127 | KM128 |
| sb + bz | bc + lc | cb + sb | cc + sc | lb + bc | lc + bz | bc + sb | cb + sc |
| KM129 | KM130 | KM131 | KM132 | KM133 | KM134 | KM135 | KM136 |
| cc + bc | lb + bz | bc + sc | cb + bc | cc + bz | bc + bc | cb + bz | bc + bz |
| KM137 | KM138 | KM139 | KM140 | KM141 | KM142 | KM143 | KM144 |
| s + h + b | s + b + u | s + u + p | s + p + pi | s + pi + e | s + e + a | s + a + bc | s + bc + cb |
| KM145 | KM146 | KM147 | KM148 | KM149 | KM150 | KM151 | KM152 |
| s + h + u | s + b + p | s + u + pi | s + p + e | s + pi + a | s + e + bc | s + a + cb | s + bc + cc |
| KM153 | KM154 | KM155 | KM156 | KM157 | KM158 | KM159 | KM160 |
| s + h + p | s + b + pi | s + u + e | s + p + a | s + pi + bc | s + e + cb | s + a + cc | s + bc + lb |
| KM161 | KM162 | KM163 | KM164 | KM165 | KM166 | KM167 | KM168 |
| s + h + pi | s + b + e | s + u + a | s + p + bc | s + pi + cb | s + e + cc | s + a + lb | s + bc + lc |
| KM169 | KM170 | KM171 | KM172 | KM173 | KM174 | KM175 | KM176 |
| s + h + e | s + b + a | s + u + bc | s + p + cb | s + pi + cc | s + e + lb | s + a + lc | s + bc + sb |
| KM177 | KM178 | KM179 | KM180 | KM181 | KM182 | KM183 | KM184 |
| s + h + a | s + b + bc | s + u + cb | s + p + cc | s + pi + lb | s + e + lc | s + a + sb | s + bc + sc |
| KM185 | KM186 | KM187 | KM188 | KM189 | KM190 | KM191 | KM192 |
| s + h + bc | s + b + cb | s + u + cc | s + p + lb | s + pi + lc | s + e + sb | s + a + sc | s + bc + bc |
| KM193 | KM194 | KM195 | KM196 | KM197 | KM198 | KM199 | KM200 |
| s + h + cb | s + b + cc | s + u + lb | s + p + lc | s + pi + sb | s + e + sc | s + a + bc | s + bc + bz |
| KM201 | KM202 | KM203 | KM204 | KM205 | KM206 | KM207 | KM208 |
| s + h + cc | s + b + lb | s + u + lc | s + p + sb | s + pi + sc | s + e + bc | s + a + bz | s + h + lb |
| KM209 | KM210 | KM211 | KM212 | KM213 | KM214 | KM215 | KM216 |
| s + b + lc | s + u + sb | s + p + sc | s + pi + bc | s + e + bz | s + h + lc | s + b + sb | s + u + sc |
| KM217 | KM218 | KM219 | KM220 | KM221 | KM222 | KM223 | KM224 |
| s + p + bc | s + pi + bz | s + h + sb | s + b + sc | s + u + bc | s + p + bz | s + h + sc | s + b + bc |
| KM225 | KM226 | KM227 | KM228 | KM229 | KM230 | KM231 | KM232 |
| s + u + bz | s + h + bc | s + b + bz | s + h + bz | s + cb + cc | s + cc + lb | s + lb + lc | s + lc + sb |
| KM233 | KM234 | KM235 | KM236 | KM237 | KM238 | KM239 | KM240 |
| s + sb + sc | s + sc + bc | s + bc + bz | s + cb + lb | s + cc + lc | s + lb + sb | s + lc + sc | s + sb + bc |
| KM241 | KM242 | KM243 | KM244 | KM245 | KM246 | KM247 | KM248 |
| s + sc + bz | s + cb + lc | s + cc + sb | s + lb + sc | s + lc + bc | s + sb + bz | s + cb + sb | s + cc + sc |
| KM249 | KM250 | KM251 | KM252 | KM253 | KM254 | KM255 | KM256 |
| s + lb + bc | s + lc + bz | s + cb + sc | s + cc + bc | s + lb + bz | s + cb + bc | s + cc + bz | s + cb + bz |
| KM257 | KM258 | KM259 | KM260 | KM261 | KM262 | KM263 | KM264 |
| h + b + u | h + u + p | h + p + pi | h + pi + e | h + e + a | h + a + bc | h + bc + cb | h + cb + cc |
| KM265 | KM266 | KM267 | KM268 | KM269 | KM270 | KM271 | KM272 |
| h + b + p | h + u + pi | h + p + e | h + pi + a | h + e + bc | h + a + cb | h + bc + cc | h + cb + lb |
| KM273 | KM274 | KM275 | KM276 | KM277 | KM278 | KM279 | KM280 |
| h + b + pi | h + u + e | h + p + a | h + pi + bc | h + e + cb | h + a + cc | h + bc + lb | h + cb + lc |
| KM281 | KM282 | KM283 | KM284 | KM285 | KM286 | KM287 | KM288 |
| h + b + e | h + u + a | h + p + bc | h + pi + cb | h + e + cc | h + a + lb | h + bc + lc | h + cb + sb |
| KM289 | KM290 | KM291 | KM292 | KM293 | KM294 | KM295 | KM296 |
| h + b + a | h + u + bc | h + p + cb | h + pi + cc | h + e + lb | h + a + lc | h + bc + sb | h + cb + sc |
| KM297 | KM298 | KM299 | KM300 | KM301 | KM302 | KM303 | KM304 |
| h + b + bc | h + u + cb | h + p + cc | h + pi + lb | h + e + lc | h + a + sb | h + bc + sc | h + cb + bc |
| KM305 | KM306 | KM307 | KM308 | KM309 | KM310 | KM311 | KM312 |
| h + b + cb | h + u + cc | h + p + lb | h + pi + lc | h + e + sb | h + a + sc | h + bc + bc | h + cb + bz |
| KM313 | KM314 | KM315 | KM316 | KM317 | KM318 | KM319 | KM320 |
| h + b + cc | h + u + lb | h + p + lc | h + pi + sb | h + e + sc | h + a + bc | h + bc + bz | h + b + lb |
| KM321 | KM322 | KM323 | KM324 | KM325 | KM326 | KM327 | KM328 |
| h + u + lc | h + p + sb | h + pi + sc | h + e + bc | h + a + bz | h + b + lc | h + u + sb | h + p + sc |
| KM329 | KM330 | KM331 | KM332 | KM333 | KM334 | KM335 | KM336 |
| h + pi + bc | h + e + bz | h + b + sb | h + u + sc | h + p + bc | h + pi + bz | h + b + sc | h + u + bc |
| KM337 | KM338 | KM339 | KM340 | KM341 | KM342 | KM343 | KM344 |
| h + p + bz | h + b + bc | h + u + bz | h + b + bz | h + cc + lb | h + lb + lc | h + lc + sb | h + sb + sc |
| KM345 | KM346 | KM347 | KM348 | KM349 | KM350 | KM351 | KM352 |
| h + sc + bc | h + bc + bz | h + cc + lc | h + lb + sb | h + lc + sc | h + sb + bc | h + sc + bz | h + cc + sb |
| KM353 | KM354 | KM355 | KM356 | KM357 | KM358 | KM359 | KM360 |
| h + lb + sc | h + lc + bc | h + sb + bz | h + cc + sc | h + lb + bc | h + lc + bz | h + cc + bc | h + lb + bz |
| KM361 | KM362 | KM363 | KM364 | KM365 | KM366 | KM367 | KM368 |
| h + cc + bz | b + u + p | b + p + pi | b + pi + e | b + e + a | b + a + bc | b + bc + cb | b + cb + cc |
| KM369 | KM370 | KM371 | KM372 | KM373 | KM374 | KM375 | KM376 |
| b + cc + lb | b + u + pi | b + p + e | b + pi + a | b + e + bc | b + a + cb | b + bc + cc | b + cb + lb |
| KM377 | KM378 | KM379 | KM380 | KM381 | KM382 | KM383 | KM384 |
| b + cc + lc | b + u + e | b + p + a | b + pi + bc | b + e + cb | b + a + cc | b + bc + lb | b + cb + lc |
| KM385 | KM386 | KM387 | KM388 | KM389 | KM390 | KM391 | KM392 |
| b + cc + sb | b + u + a | b + p + bc | b + pi + cb | b + e + cc | b + a + lb | b + bc + lc | b + cb + sb |
| KM393 | KM394 | KM395 | KM396 | KM397 | KM398 | KM399 | KM400 |
| b + cc + sc | b + u + bc | b + p + cb | b + pi + cc | b + e + lb | b + a + lc | b + bc + sb | b + cb + sc |
| KM401 | KM402 | KM403 | KM404 | KM405 | KM406 | KM407 | KM408 |
| b + cc + bc | b + u + cb | b + p + cc | b + pi + lb | b + e + lc | b + a + sb | b + bc + sc | b + cb + bc |
| KM409 | KM410 | KM411 | KM412 | KM413 | KM414 | KM415 | KM416 |
| b + cc + bz | b + u + cc | b + p + lb | b + pi + lc | b + e + sb | b + a + sc | b + bc + bc | b + cb + bz |
| KM417 | KM418 | KM419 | KM420 | KM421 | KM422 | KM423 | KM424 |
| b + u + lb | b + p + lc | b + pi + sb | b + e + sc | b + a + bc | b + bc + bz | b + u + lc | b + p + sb |
| KM425 | KM426 | KM427 | KM428 | KM429 | KM430 | KM431 | KM432 |
| b + pi + sc | b + e + bc | b + a + bz | b + u + sb | b + p + sc | b + pi + bc | b + e + bz | b + u + sc |
| KM433 | KM434 | KM435 | KM436 | KM437 | KM438 | KM439 | KM440 |
| b + p + bc | b + pi + bz | b + u + bc | b + p + bz | b + u + bz | b + lb + lc | b + lc + sb | b + sb + sc |
| KM441 | KM442 | KM443 | KM444 | KM445 | KM446 | KM447 | KM448 |
| b + sc + bc | b + bc + bz | b + lb + sb | b + lc + sc | b + sb + bc | b + sc + bz | b + lb + sc | b + lc + bc |
| KM449 | KM450 | KM451 | KM452 | KM453 | KM454 | KM455 | KM456 |
| b + sb + bz | b + lb + bc | b + lc + bz | b + lb + bz | u + p + pi | u + pi + e | u + e + a | u + a + bc |
| KM457 | KM458 | KM459 | KM460 | KM461 | KM462 | KM463 | KM464 |
| u + bc + cb | u + cb + cc | u + cc + lb | u + lb + lc | u + p + e | u + pi + a | u + e + bc | u + a + cb |
| KM465 | KM466 | KM467 | KM468 | KM469 | KM470 | KM471 | KM472 |
| u + bc + cc | u + cb + lb | u + cc + lc | u + lb + sb | u + p + a | u + pi + bc | u + e + cb | u + a + cc |
| KM473 | KM474 | KM475 | KM476 | KM477 | KM478 | KM479 | KM480 |
| u + bc + lb | u + cb + lc | u + cc + sb | u + lb + sc | u + p + bc | u + pi + cb | u + e + cc | u + a + lb |
| KM481 | KM482 | KM483 | KM484 | KM485 | KM486 | KM487 | KM488 |
| u + bc + lc | u + cb + sb | u + cc + sc | u + lb + bc | u + p + cb | u + pi + cc | u + e + lb | u + a + lc |
| KM489 | KM490 | KM491 | KM492 | KM493 | KM494 | KM495 | KM496 |
| u + bc + sb | u + cb + sc | u + cc + bc | u + lb + bz | u + p + cc | u + pi + lb | u + e + lc | u + a + sb |
| KM497 | KM498 | KM499 | KM500 | KM501 | KM502 | KM503 | KM504 |
| u + bc + sc | u + cb + bc | u + cc + bz | u + p + lb | u + pi + lc | u + e + sb | u + a + sc | u + bc + bc |
| KM505 | KM506 | KM507 | KM508 | KM509 | KM510 | KM511 | KM512 |
| u + cb + bz | u + p + lc | u + pi + sb | u + e + sc | u + a + bc | u + bc + bz | u + p + sb | u + pi + sc |
| KM513 | KM514 | KM515 | KM516 | KM517 | KM518 | KM519 | KM520 |
| u + e + bc | u + a + bz | u + p + sc | u + pi + bc | u + e + bz | u + p + bc | u + pi + bz | u + p + bz |
| KM521 | KM522 | KM523 | KM524 | KM525 | KM526 | KM527 | KM528 |
| p + pi + e | p + e + a | p + a + bc | p + bc + cb | p + cb + cc | p + cc + lb | p + lb + lc | p + lc + sb |
| KM529 | KM530 | KM531 | KM532 | KM533 | KM534 | KM535 | KM536 |
| p + pi + a | p + e + bc | p + a + cb | p + bc + cc | p + cb + lb | p + cc + lc | p + lb + sb | p + lc + sc |
| KM537 | KM538 | KM539 | KM540 | KM541 | KM542 | KM543 | KM544 |
| p + pi + bc | p + e + cb | p + a + cc | p + bc + lb | p + cb + lc | p + cc + sb | p + lb + sc | p + lc + bc |
| KM545 | KM546 | KM547 | KM548 | KM549 | KM550 | KM551 | KM552 |
| p + pi + cb | p + e + cc | p + a + lb | p + bc + lc | p + cb + sb | p + cc + sc | p + lb + bc | p + lc + bz |
| KM553 | KM554 | KM555 | KM556 | KM557 | KM558 | KM559 | KM560 |
| p + pi + cc | p + e + lb | p + a + lc | p + bc + sb | p + cb + sc | p + cc + bc | p + lb + bz | p + pi + lb |
| KM561 | KM562 | KM563 | KM564 | KM565 | KM566 | KM567 | KM568 |
| p + e + lc | p + a + sb | p + bc + sc | p + cb + bc | p + cc + bz | p + pi + lc | p + e + sb | p + a + sc |
| KM569 | KM570 | KM571 | KM572 | KM573 | KM574 | KM575 | KM576 |
| p + bc + bc | p + pi + bc | p + e + bz | p + e + sc | p + a + bc | p + bc + bz | p + pi + sc | p + e + bc |
| KM577 | KM578 | KM579 | KM580 | KM581 | KM582 | KM583 | KM584 |
| p + a + bz | p + pi + bz | u + lc + sb | u + sb + sc | u + sc + bc | u + bc + bz | u + lc + sc | u + sb + bc |
| KM585 | KM586 | KM587 | KM588 | KM589 | KM590 | KM591 | KM592 |
| u + sc + bz | u + lc + bc | u + sb + bz | u + lc + bz | p + sb + sc | p + sc + bc | p + bc + bz | p + sb + bc |
| KM593 | KM594 | KM595 | KM596 | KM597 | KM598 | KM599 | KM600 |
| p + sc + bz | p + sb + bz | pi + e + a | pi + a + bc | pi + bc + cb | pi + cb + cc | pi + cc + lb | pi + lb + lc |
| KM601 | KM602 | KM603 | KM604 | KM605 | KM606 | KM607 | KM608 |
| pi + lc + sb | pi + sb + sc | pi + e + bc | pi + a + cb | pi + bc + cc | pi + cb+ lb | pi + cc + lc | pi + lb + sb |
| KM609 | KM610 | KM611 | KM612 | KM613 | KM614 | KM615 | KM616 |
| pi + lc + sc | pi + sb + bc | pi + e + cb | pi + a + cc | pi + bc + lb | pi + cb + lc | pi + cc + sb | pi + lb + sc |
| KM625 | KM626 | KM627 | KM628 | KM629 | KM630 | KM631 | KM632 |
| pi + lc + bc | pi + sb + bz | pi + e + cc | pi + a + lb | pi + bc + lc | pi + cb + sb | pi + cc + sc | pi + lb + bc |
| KM633 | KM634 | KM635 | KM636 | KM637 | KM638 | KM639 | KM640 |
| pi + lc + bz | pi + e + lb | pi + a + lc | pi + bc + sb | pi + cb + sc | pi + cc + bc | pi + lb + bz | pi + e + lc |
| KM641 | KM642 | KM643 | KM644 | KM645 | KM646 | KM647 | KM648 |
| pi + a + sb | pi + bc + sc | pi + cb + bc | pi + cc + bz | pi + e + sb | pi + a + sc | pi + bc + bc | pi + cb + bz |
| KM649 | KM650 | KM651 | KM652 | KM653 | KM654 | KM655 | KM656 |
| pi + e + sc | pi + a + bc | pi + bc + bz | pi + e + bc | pi + a + bz | pi + e + bz | pi + sc + bc | pi + bc + bz |
| KM657 | KM658 | KM659 | KM660 | KM661 | KM662 | KM663 | KM664 |
| pi + sc + bz | e + a + bc | e + bc + cb | e + cb + cc | e + cc + lb | e + lb + lc | e + lc + sb | e + sb + sc |
| KM665 | KM666 | KM667 | KM668 | KM669 | KM670 | KM671 | KM672 |
| e + sc + bc | e + a + cb | e + bc + cc | e + cb + lb | e + cc + lc | e + lb + sb | e + lc + sc | e + sb + bc |
| KM673 | KM674 | KM675 | KM676 | KM677 | KM678 | KM679 | KM680 |
| e + sc + bz | e + a + cc | e + bc + lb | e + cb + lc | e + cc + sb | e + lb + sc | e + lc + bc | e + sb + bz |
| KM681 | KM682 | KM683 | KM684 | KM685 | KM686 | KM687 | KM688 |
| e + a + lb | e + bc + lc | e + cb + sb | e + cc + sc | e + lb + bc | e + lc + bz | e + a + lc | e + bc + sb |
| KM689 | KM690 | KM691 | KM692 | KM693 | KM694 | KM695 | KM696 |
| e + cb + sc | e + cc + bc | e + lb + bz | e + a + sb | e + bc + sc | e + cb + bc | e + cc + bz | e + a + sc |
| KM697 | KM698 | KM699 | KM700 | KM701 | KM702 | KM703 | KM704 |
| e + bc + bc | e + cb + bz | e + a + bc | e + bc + bz | e + a + bz | e + bc + bz | a + bc + cb | a + cb + cc |
| KM705 | KM706 | KM707 | KM708 | KM709 | KM710 | KM711 | KM712 |
| a + cc + lb | a + lb + lc | a + lc + sb | a + sb + sc | a + sc + bc | a + bc + bz | a + bc + cc | a + cb + lb |
| KM713 | KM714 | KM715 | KM716 | KM717 | KM718 | KM719 | KM720 |
| a + cc + lc | a + lb + sb | a + lc + sc | a + sb + bc | a + sc + bz | a + bc + lb | a + cb + lc | a + cc + sb |
| KM721 | KM722 | KM723 | KM724 | KM725 | KM726 | KM727 | KM728 |
| a + lb + sc | a + lc + bc | a + sb + bz | a + bc + lc | a + cb + sb | a + cc + sc | a + lb + bc | a + lc + bz |
| KM729 | KM730 | KM731 | KM732 | KM733 | KM734 | KM735 | KM736 |
| a + bc + sb | a + cb + sc | a + cc + bc | a + lb + bz | a + bc + sc | a + cb + bc | a + cc + bz | a + bc + bc |
| KM737 | KM738 | KM739 | KM740 | KM741 | KM742 | KM743 | KM744 |
| a + cb + bz | a + bc + bz | bc + cb + | bc + cc + lb | bc + lb + lc | bc + lc + sb | bc + sb + | bc + sc + bc |
| | | cc | | | | sc | |
| KM745 | KM746 | KM747 | KM748 | KM749 | KM750 | KM751 | KM752 |
| bc + bc + | bc + cb + lb | bc + cc + lc | bc + lb + sb | bc + lc + sc | bc + sb + | bc + sc + | bc + cb + lc |
| bz | | | | | bc | bz | |
| KM753 | KM754 | KM755 | KM756 | KM757 | KM758 | KM759 | KM760 |
| bc + cc + sb | bc + lb + sc | bc + lc + bc | bc + sb + | bc + cb + | bc + cc + sc | bc + lb + bc | bc + lc + bz |
| | | | bz | sb | | | |
| KM761 | KM762 | KM763 | KM764 | KM765 | KM766 | KM767 | KM768 |
| bc + cb + sc | bc + cc + | bc + lb + bz | bc + cb + | bc + cc + bz | bc + cb + | cb + cc + lb | cb + lb + lc |
| | bc | | bc | | bz | | |
| KM769 | KM770 | KM771 | KM772 | KM773 | KM774 | KM775 | KM776 |
| cb + lc + sb | cb + sb + | cb + sc + | cb + bc + | cb + cc + lc | cb + lb + sb | cb + lc + sc | cb + sb + |
| | sc | bc | bz | | | | bc |
| KM777 | KM778 | KM779 | KM780 | KM781 | KM782 | KM783 | KM784 |
| cb + sc + bz | cb + cc + | cb + lb + sc | cb + lc + bc | cb + sb + | cb + cc + sc | cb + lb + bc | cb + lc + bz |
| | sb | | | bz | | | |
| KM785 | KM786 | KM787 | KM788 | KM789 | KM790 | KM791 | KM792 |
| cb + cc + bc | cb + lb + bz | cb + cc + | cc + lb + lc | cc + lc + sb | cc + sb + sc | cc + sc + bc | cc + bc + bz |
| | | bz | | | | | |
| KM793 | KM794 | KM795 | KM796 | KM797 | KM798 | KM799 | KM800 |
| cc + lb + sb | cc + lc + sc | cc + sb + | cc + sc + bz | cc + lb + sc | cc + lc + bc | cc + sb + | cc + lb + bc |
| | | bc | | | | bz | |
| KM801 | KM802 | KM803 | KM804 | KM805 | KM806 | KM807 | KM808 |
| cc + lc + bz | cc + lb + bz | lb + lc + sb | lb + sb + sc | lb + sc + bc | lb + bc + bz | lb + lc + sc | lb + sb + bc |
| KM809 | KM810 | KM811 | KM812 | KM813 | KM814 | KM815 | KM816 |
| lb + sc + bz | lb + lc + bc | lb + sb + bz | lb + lc + bz | lc + sb + sc | lc + sc + bc | lc + bc + bz | lc + sb + bc |
| KM817 | KM818 | KM819 | KM820 | KM821 | KM822 | KM823 | KM824 |
| lc + sc + bz | lc + sb + bz | sb + sc + | sb + bc + | sb + sc + | sc + bc + bz | sb + aq + oc | sc + aq + |
| | | bc | bz | bz | | | oc |
| KM825 | KM826 | KM827 | KM828 | KM829 | KM830 | KM831 | KM832 |
| s + h + b + u | s + h + u + p | s + h + p + pi | s + h + pi + e | s + h + a + | s + b + u + p | s + b + p + pi | s + b + pi + e |
| | | | | aq | | | |
| KM833 | KM834 | KM835 | KM836 | KM837 | KM838 | KM839 | KM840 |
| s + h + b + p | s + h + u + pi | s + h + p + e | s + h + pi + a | s + h + u + | s + b + u + pi | s + b + p + e | s + b + pi + a |
| | | | | aq | | | |
| KM841 | KM842 | KM843 | KM844 | KM845 | KM846 | KM847 | KM848 |
| s + h + b + pi | s + h + u + e | s + h + p + a | s + h + pi + | s + h + b + a | s + b + u + e | s + b + p + a | s + b + pi + |
| | | | aq | | | | aq |
| KM849 | KM850 | KM851 | KM852 | KM853 | KM854 | KM855 | KM856 |
| s + h + b + e | s + h + u + a | s + h + p + | s + b + u + a | s + b + p + | s + b + u + | s + h + b + | s + b + e + a |
| | | aq | | aq | aq | aq | |
| KM857 | KM858 | KM859 | KM860 | KM861 | KM862 | KM863 | KM864 |
| s + b + a + | s + u + p + pi | s + u + pi + e | s + u + e + a | s + u + a + | s + b + e + | s + u + p + e | s + u + pi + a |
| aq | | | | aq | aq | | |
| KM865 | KM866 | KM867 | KM868 | KM869 | KM870 | KM871 | KM872 |
| s + u + e + | s + u + p + a | s + u + pi + | s + u + p + | s + p + pi + e | s + p + e + a | s + p + a + aq | s + pi + e + a |
| aq | | aq | aq | | | | |
| KM873 | KM874 | KM875 | KM876 | KM877 | KM878 | KM879 | KM880 |
| s + pi + a + | s + e + a + | s + p + pi + a | s + p + e + | s + pi + e + | s + p + pi + | h + b + u + p | h + b + p + |
| aq | aq | | aq | aq | aq | | pi |
| KM881 | KM882 | KM883 | KM884 | KM885 | KM886 | KM887 | KM888 |
| h + b + pi + e | h + b + e + a | h + b + a + aq | h + u + p + | h + u + pi + e | h + u + e + a | h + b + u + | h + b + p + e |
| | | | pi | | | pi | |
| KM889 | KM890 | KM891 | KM892 | KM893 | KM894 | KM895 | KM896 |
| h + b + pi + a | h + b + e + | h + u + p + e | h + u + pi + a | h + u + e + | h + b + u + e | h + b + p + a | h + b + pi + |
| | aq | | | aq | | | aq |
| KM897 | KM898 | KM899 | KM900 | KM901 | KM902 | KM903 | KM904 |
| h + u + p + a | h + u + pi + | h + b + u + a | h + b + p + | h + u + p + | h + b + u + | h + u + a + | h + p + pi + e |
| | aq | | aq | aq | aq | aq | |
| KM905 | KM906 | KM907 | KM908 | KM909 | KM910 | KM911 | KM912 |
| h + p + e + a | h + p + a + aq | h + pi + e + a | h + pi + e + | h + e + a + aq | h + p + pi + a | h + p + e + | h + pi + e + |
| | | | aq | | | aq | aq |
| KM913 | KM914 | KM915 | KM916 | KM917 | KM918 | KM919 | KM920 |
| h + p + pi + | b + u + p + | b + u + pi + e | b + u + e + a | b + u + a + | b + p + pi + e | b + p + e + a | b + p + a + |
| aq | pi | | | aq | | | aq |
| KM921 | KM922 | KM923 | KM924 | KM925 | KM926 | KM927 | KM928 |
| b + pi + e + a | b + pi + a + | b + e + a + | b + u + p + e | b + u + pi + a | b + u + e + | b + p + pi + a | b + p + e + |
| | aq | aq | | | aq | | aq |
| KM929 | KM930 | KM931 | KM932 | KM933 | KM934 | KM935 | KM936 |
| b + pi + e + | b + u + p + a | b + u + pi + | b + p + pi + | b + u + p + | u + p + pi + e | u + p + e + a | u + p + a + |
| aq | | aq | aq | aq | | | aq |
| KM937 | KM938 | KM939 | KM940 | KM941 | KM942 | KM943 | KM944 |
| u + pi + e + a | u + pi + a + | u + e + a + | u + p + pi + a | u + p + e + | u + pi + e + | u + p + pi + | p + pi + e + a |
| | aq | aq | | aq | aq | aq | |
| KM945 | KM946 | KM947 | KM948 | KM949 | KM950 | KM951 | KM952 |
| p + pi + a + | p + pi + e + | pi + e + a + | s + h + b + u + | s + h + b + u + | s + oc | h + oc | b + oc |
| aq | aq | aq | p + pi + a + | p + pi + a + | | | |
| | | | e + aq + oc | e + bc + cb + | | | |
| | | | | cc + lb + | | | |
| | | | | lc + sb + sc + | | | |
| | | | | bc + bz + | | | |
| | | | | oc | | | |
| KM953 | KM954 | KM955 | KM956 | KM957 | KM958 | KM959 | KM960 |
| u + oc | p + oc | pi + oc | e + oc | a + oc | bz + oc | bc + oc | cb + oc |
| KM961 | KM962 | KM963 | KM964 | KM965 | KM966 | KM967 | KM968 |
| cc + oc | lb + oc | lc + oc | sb + oc | sc + oc | aq + oc | s + h + oc | s + b + oc |
| KM969 | KM970 | KM971 | KM972 | KM973 | KM974 | KM975 | KM976 |
| s + u + oc | s + p + oc | s + pi + oc | s + e + oc | s + a + oc | s + bz + oc | s + bc + oc | s + bc + oc |
| KM977 | KM978 | KM979 | KM980 | KM981 | KM982 | KM983 | KM984 |
| s + cb + oc | s + cc + oc | s + lb + oc | s + lc + oc | s + sb + oc | s + sc + oc | s + aq + oc | h + b + oc |
| KM985 | KM986 | KM987 | KM988 | KM989 | KM990 | KM991 | KM992 |
| h + u + oc | h + p + oc | h + pi + oc | h + e + oc | h + a + oc | h + bz + oc | h + bc + oc | h + bc + oc |
| KM993 | KM994 | KM995 | KM996 | KM997 | KM998 | KM999 | KM1000 |
| h + cb + oc | h + cc + oc | h + lb + oc | h + lc + oc | h + sb + oc | h + sc + oc | h + aq + oc | u + p + oc |
| KM1001 | KM1002 | KM1003 | KM1004 | KM1005 | KM1006 | KM1007 | KM1008 |
| u + pi + oc | u + e + oc | u + a + oc | u + bz + oc | u + bc + oc | u + bc + oc | u + cb + oc | u + cc + oc |
| KM1009 | KM1010 | KM1011 | KM1012 | KM1013 | KM1014 | KM1015 | KM1016 |
| u + lb + oc | u + lc + oc | u + sb + oc | u + sc + oc | u + aq + oc | p + pi + oc | p + e + oc | p + a + oc |
| KM1017 | KM1018 | KM1019 | KM1020 | KM1021 | KM1022 | KM1023 | KM1024 |
| p + bz + oc | p + bc + oc | p + cb + oc | p + cc + oc | p + lb + oc | p + lc + oc | p + sb + oc | p + sc + oc |
| KM1025 | KM1026 | KM1027 | KM1028 | KM1029 | KM1030 | KM1031 | KM1032 |
| pi + e + oc | pi + a + oc | pi + bz + oc | pi + bc + oc | pi + bc + oc | pi + cb + oc | pi + cc + oc | pi + lb + oc |
| KM1033 | KM1034 | KM1035 | KM1036 | KM1037 | KM1038 | KM1039 | KM1040 |
| pi + lc + oc | pi + sb + | pi + sc + oc | pi + aq + oc | e + a + oc | e + bz + oc | e + bc + oc | e + cb + oc |
| | oc | | | | | | |
| KM1041 | KM1042 | KM1043 | KM1044 | KM1045 | KM1046 | KM1047 | KM1048 |
| e + cc + oc | e + lb + oc | e + lc + oc | e + sb + oc | e + sc + oc | e + aq + oc | a + bz + oc | a + bc + oc |
| KM1049 | KM1050 | KM1051 | KM1052 | KM1053 | KM1054 | KM1055 | KM1056 |
| a + bc + oc | a + cb + oc | a + cc + oc | a + lb + oc | a + lc + oc | a + sb + oc | a + sc + oc | p + aq + oc |
| KM1057 | KM1058 | KM1059 | KM1060 | KM1061 | KM1062 | KM1063 | KM1064 |
| a + aq + | bz + bc + | bz + cb + oc | bz + cc + oc | bz + lb + oc | bz + lc + oc | bz + sb + oc | bz + sc + |
| oc | oc | | | | | | oc |
| KM1065 | KM1066 | KM1067 | KM1068 | KM1069 | KM1070 | KM1071 | KM1072 |
| bz + aq + oc | bc + cb + | bc + cc + oc | bc + lb + oc | bc + lc + oc | bc + sb + oc | bc + sc + oc | bc + aq + |
| | oc | | | | | | oc |
| KM1073 | KM1074 | KM1075 | KM1076 | KM1077 | KM1078 | KM1079 | KM1080 |
| cb + cc + | cb + lb + | cb + lc + oc | cb + sb + oc | cb + sc + oc | cb + aq + oc | cc + lb + oc | cc + lc + oc |
| oc | oc | | | | | | |
| KM1081 | KM1082 | KM1083 | KM1084 | KM1085 | KM1086 | KM1087 | KM1088 |
| cc + sb + | cc + sc + | cc + aq + oc | lb + lc + oc | lb + sb + oc | lb + sc + oc | lb + aq + oc | lc + sb + oc |
| oc | oc | | | | | | |
| KM1089 | KM1090 | KM1091 | KM1092 | KM1093 | KM1094 | KM1095 | KM1096 |
| lc + sc + | lc + aq + | sb + sc + oc | s + h + b + oc | s + b + u + oc | s + u + p + oc | s + p + pi + | s + pi + e + |
| oc | oc | | | | | oc | oc |
| KM1097 | KM1098 | KM1099 | KM1100 | KM1101 | KM1102 | KM1103 | KM1104 |
| s + e + a + oc | s + a + aq + | s + h + a + | s + h + u + | s + h + oc + | s + h + pi + | s + h + p + | s + b + p + |
| | oc | oc | oc | oc | oc | oc | oc |
| KM1105 | KM1106 | KM1107 | KM1108 | KM1109 | KM1110 | KM1111 | KM1112 |
| s + b + u + | s + h + b + | s + b + a + | s + u + a + | s + b + e + | s + u + e + | s + u + pi + | s + u + p + |
| oc | oc | oc | oc | oc | oc | oc | oc |
| KM1113 | KM1114 | KM1115 | KM1116 | KM1117 | KM1118 | KM1119 | KM1120 |
| s + p + a + oc | s + pi + a + | s + e + a + | s + p + e + | s + pi + e + | s + p + pi + | h + b + a + | h + b + e + |
| | oc | oc | oc | oc | oc | oc | oc |
| KM1121 | KM1122 | KM1123 | KM1124 | KM1125 | KM1126 | KM1127 | KM1128 |
| h + u + e + | h + b + pi + | h + u + pi + | h + b + p + | h + u + p + | h + b + u + | h + u + a + | h + p + a + |
| oc | oc | oc | oc | oc | oc | oc | oc |
| KM1129 | KM1130 | KM1131 | KM1132 | KM1133 | KM1134 | KM1135 | KM1136 |
| h + pi + e + | h + e + a + oc | h + p + e + | h + pi + e + | h + p + pi + | b + u + a + | b + p + a + | b + pi + e + |
| oc | | oc | oc | oc | oc | oc | oc |
| KM1137 | KM1138 | KM1139 | KM1140 | KM1141 | KM1142 | KM1143 | KM1144 |
| b + u + pi + | b + p + pi + | b + u + p + | u + p + a + | u + pi + a + | u + e + a + | u + p + e + | u + pi + e + |
| oc | oc | oc | oc | oc | oc | oc | oc |
| KM1145 | KM1146 | KM1147 | KM1148 | KM1149 | KM1150 | KM1151 | KM1152 |
| u + p + pi + | p + pi + a + | p + pi + e + | pi + e + a + | e + a + aq + | s + h + b + u + | s + h + b + u + | s + h + b + u + |
| oc | oc | oc | oc | oc | p + pi + a + | p + pi + a + | p + pi + a + |
| | | | | | e + aq + oc + | e + aq + oc + | e + bc + cb + |
| | | | | | hd | hp | cc + lb + |
| | | | | | | | lc + sb + |
| | | | | | | | sc + bc + bz + |
| | | | | | | | oc + hd |
| KM1153 | KM1154 | KM1155 | KM1156 | KM1157 | KM1158 | KM1159 | KM1160 |
| s + h + b + u + | s + hx | h + hx | b + hx | s + hd | h + hd | b + hd | s + hp |
| p + pi + a + | | | | | | | |
| e + bc + cb + | | | | | | | |
| cc + lb + | | | | | | | |
| lc + sb + | | | | | | | |
| sc + bc + bz + | | | | | | | |
| oc + | | | | | | | |
| hp | | | | | | | |
| KM1161 | KM1162 | KM1163 | KM1164 | KM1165 | KM1166 | KM1167 | KM1168 |
| u + hx | p + hx | pi + hx | e + hx | a + hx | bz + hx | bc + hx | cb + hx |
| KM1169 | KM1170 | KM1171 | KM1172 | KM1173 | KM1174 | KM1175 | KM1176 |
| cc + hx | lb + hx | lc + hx | sb + hx | sc + hx | aq + hx | s + h + hx | s + b + hx |
| KM1177 | KM1178 | KM1179 | KM1180 | KM1181 | KM1182 | KM1183 | KM1184 |
| s + u + hx | s + p + hx | s + pi + hx | s + e + hx | s + a + hx | s + bz + hx | s + bc + hx | s + bc + hx |
| KM1185 | KM1186 | KM1187 | KM1188 | KM1189 | KM1190 | KM1191 | KM1192 |
| s + cb + hx | s + cc + hx | s + lb + hx | s + lc + hx | s + sb + hx | s + sc + hx | s + aq + hx | h + b + hx |
| KM1193 | KM1194 | KM1195 | KM1196 | KM1197 | KM1198 | KM1199 | KM1200 |
| h + u + hx | h + p + hx | h + pi + hx | h + e + hx | h + a + hx | h + bz + hx | h + bc + hx | h + bc + hx |
| KM1201 | KM1202 | KM1203 | KM1204 | KM1205 | KM1206 | KM1207 | KM1208 |
| h + cb + hx | h + cc + hx | h + lb + hx | h + lc + hx | h + sb + hx | h + sc + hx | h + aq + hx | u + p + hx |
| KM1209 | KM1210 | KM1211 | KM1212 | KM1213 | KM1214 | KM1215 | KM1216 |
| u + pi + hx | u + e + hx | u + a + hx | u + bz + hx | u + bc + hx | u + bc + hx | u + cb + hx | u + cc + hx |
| KM1217 | KM1218 | KM1219 | KM1220 | KM1221 | KM1222 | KM1223 | KM1224 |
| u + lb + hx | u + lc + hx | u + sb + hx | u + sc + hx | u + aq + hx | p + pi + hx | p + e + hx | p + a + hx |
| KM1225 | KM1226 | KM1227 | KM1228 | KM1229 | KM1230 | KM1231 | KM1232 |
| p + bz + hx | p + bc + hx | p + cb + hx | p + cc + hx | p + lb + hx | p + lc + hx | p + sb + hx | p + sc + hx |
| KM1233 | KM1234 | KM1235 | KM1236 | KM1237 | KM1238 | KM1239 | KM1240 |
| pi + e + hx | pi + a + hx | pi + bz + hx | pi + bc + hx | pi + bc + hx | pi + cb + hx | pi + cc + hx | pi + lb + hx |
| KM1241 | KM1242 | KM1243 | KM1244 | KM1245 | KM1246 | KM1247 | KM1248 |
| pi + lc + hx | pi + sb + | pi + sc + hx | pi + aq + hx | e + a + hx | e + bz + hx | e + bc + hx | e + cb + hx |
| | hx | | | | | | |
| KM1249 | KM1250 | KM1251 | KM1252 | KM1253 | KM1254 | KM1255 | KM1256 |
| e + cc + hx | e + lb + hx | e + lc + hx | e + sb + hx | e + sc + hx | e + aq + hx | a + bz + hx | a + bc + hx |
| KM1257 | KM1258 | KM1259 | KM1260 | KM1261 | KM1262 | KM1263 | KM1264 |
| a + bc + hx | a + cb + hx | a + cc + hx | a + lb + hx | a + lc + hx | a + sb + hx | a + sc + hx | p + aq + hx |
| KM1265 | KM1266 | KM1267 | KM1268 | KM1269 | KM1270 | KM1271 | KM1272 |
| a + aq + | bz + bc + | bz + cb + hx | bz + cc + hx | bz + lb + hx | bz + lc + hx | bz + sb + hx | bz + sc + |
| hx | hx | | | | | | hx |
| KM1273 | KM1274 | KM1275 | KM1276 | KM1277 | KM1278 | KM1279 | KM1280 |
| bz + aq + hx | bc + cb + | bc + cc + hx | bc + lb + hx | bc + lc + hx | bc + sb + hx | bc + sc + hx | bc + aq + |
| | hx | | | | | | hx |
| KM1281 | KM1282 | KM1283 | KM1284 | KM1285 | KM1286 | KM1287 | KM1288 |
| cb + cc + | cb + lb + | cb + lc + hx | cb + sb + hx | cb + sc + hx | cb + aq + hx | cc + lb + hx | cc + lc + hx |
| hx | hx | | | | | | |
| KM1289 | KM1290 | KM1291 | KM1292 | KM1293 | KM1294 | KM1295 | KM1296 |
| cc + sb + | cc + sc + | cc + aq + hx | lb + lc + hx | lb + sb + hx | lb + sc + hx | lb + aq + hx | lc + sb + hx |
| hx | hx | | | | | | |
| KM1297 | KM1298 | KM1299 | KM1300 | KM1301 | KM1302 | KM1303 | KM1304 |
| lc + sc + | lc + aq + | sb + sc + hx | s + h + b + hx | s + b + u + hx | s + u + p + hx | s + p + pi + | s + pi + e + |
| hx | hx | | | | | hx | hx |
| KM1305 | KM1306 | KM1307 | KM1308 | KM1309 | KM1310 | KM1311 | KM1312 |
| s + e + a + hx | s + a + aq + | s + h + a + | s + h + u + | s + h + hx + | s + h + pi + | s + h + p + | s + b + p + |
| | hx | hx | hx | hx | hx | hx | hx |
| KM1313 | KM1314 | KM1315 | KM1316 | KM1317 | KM1318 | KM1319 | KM1320 |
| s + b + u + | s + h + b + | s + b + a + | s + u + a + | s + b + e + | s + u + e + | s + u + pi + | s + u + p + |
| hx | hx | hx | hx | hx | hx | hx | hx |
| KM1321 | KM1322 | KM1323 | KM1324 | KM1325 | KM1326 | KM1327 | KM1328 |
| s + p + a + hx | s + pi + a + | s + e + a + | s + p + e + | s + pi + e + | s + p + pi + | h + b + a + | h + b + e + |
| | hx | hx | hx | hx | hx | hx | hx |
| KM1329 | KM1330 | KM1331 | KM1332 | KM1333 | KM1334 | KM1335 | KM1336 |
| h + u + e + | h + b + pi + | h + u + pi + | h + b + p + | h + u + p + | h + b + u + | h + u + a + | h + p + a + |
| hx | hx | hx | hx | hx | hx | hx | hx |
| KM1337 | KM1338 | KM1339 | KM1340 | KM1341 | KM1342 | KM1343 | KM1344 |
| h + pi + e + | h + e + a + hx | h + p + e + | h + pi + e + | h + p + pi + | b + u + a + | b + p + a + | b + pi + e + |
| hx | | hx | hx | hx | hx | hx | hx |
| KM1345 | KM1346 | KM1347 | KM1348 | KM1349 | KM1350 | KM1351 | KM1352 |
| b + u + pi + | b + p + pi + | b + u + p + | u + p + a + | u + pi + a + | u + e + a + | u + p + e + | u + pi + e + |
| hx | hx | hx | hx | hx | hx | hx | hx |
| KM1353 | KM1354 | KM1355 | KM1356 | KM1357 | KM1358 | KM1359 | KM1360 |
| u + p + pi + | p + pi + a + | p + pi + e + | pi + e + a + | e + a + aq + | sb + aq + hx | sc + aq + | aq + oc + |
| hx | hx | hx | hx | hx | | hx | hx |
| KM1361 | KM1362 | KM1363 | KM1364 | KM1365 | KM1366 | KM1367 | KM1368 |
| u + hd | p + hd | pi + hd | e + hd | a + hd | bz + hd | bc + hd | cb + hd |
| KM1369 | KM1370 | KM1371 | KM1372 | KM1373 | KM1374 | KM1375 | KM1376 |
| cc + hd | lb + hd | lc + hd | sb + hd | sc + hd | aq + hd | s + h + hd | s + b + hd |
| KM1377 | KM1378 | KM1379 | KM1380 | KM1381 | KM1382 | KM1383 | KM1384 |
| s + u + hd | s + p + hd | s + pi + hd | s + e + hd | s + a + hd | s + bz + hd | s + bc + hd | s + bc + hd |
| KM1385 | KM1386 | KM1387 | KM1388 | KM1389 | KM1390 | KM1391 | KM1392 |
| s + cb + hd | s + cc + hd | s + lb + hd | s + lc + hd | s + sb + hd | s + sc + hd | s + aq + hd | h + b + hd |
| KM1393 | KM1394 | KM1395 | KM1396 | KM1397 | KM1398 | KM1399 | KM1400 |
| h + u + hd | h + p + hd | h + pi + hd | h + e + hd | h + a + hd | h + bz + hd | h + bc + hd | h + bc + hd |
| KM1401 | KM1402 | KM1403 | KM1404 | KM1405 | KM1406 | KM1407 | KM1408 |
| h + cb + | h + cc + hd | h + lb + hd | h + lc + hd | h + sb + hd | h + sc + hd | h + aq + hd | u + p + hd |
| hd | | | | | | | |
| KM1409 | KM1410 | KM1411 | KM1412 | KM1413 | KM1414 | KM1415 | KM1416 |
| u + pi + hd | u + e + hd | u + a + hd | u + bz + hd | u + bc + hd | u + bc + hd | u + cb + hd | u + cc + hd |
| KM1417 | KM1418 | KM1419 | KM1420 | KM1421 | KM1422 | KM1423 | KM1424 |
| u + lb + hd | u + lc + hd | u + sb + hd | u + sc + hd | u + aq + hd | p + pi + hd | p + e + hd | p + a + hd |
| KM1425 | KM1426 | KM1427 | KM1428 | KM1429 | KM1430 | KM1431 | KM1432 |
| p + bz + hd | p + bc + hd | p + cb + hd | p + cc + hd | p + lb + hd | p + lc + hd | p + sb + hd | p + sc + hd |
| KM1433 | KM1434 | KM1435 | KM1436 | KM1437 | KM1438 | KM1439 | KM1440 |
| pi + e + hd | pi + a + hd | pi + bz + hd | pi + bc + hd | pi + bc + hd | pi + cb + hd | pi + cc + hd | pi + lb + hd |
| KM1441 | KM1442 | KM1443 | KM1444 | KM1445 | KM1446 | KM1447 | KM1448 |
| pi + lc + hd | pi + sb + | pi + sc + hd | pi + aq + hd | e + a + hd | e + bz + hd | e + bc + hd | e + cb + hd |
| | hd | | | | | | |
| KM1449 | KM1450 | KM1451 | KM1452 | KM1453 | KM1454 | KM1455 | KM1456 |
| e + cc + hd | e + lb + hd | e + lc + hd | e + sb + hd | e + sc + hd | e + aq + hd | a + bz + hd | a + bc + hd |
| KM1457 | KM1458 | KM1459 | KM1460 | KM1461 | KM1462 | KM1463 | KM1464 |
| a + bc + | a + cb + | a + cc + hd | a + lb + hd | a + lc + hd | a + sb + hd | a + sc + hd | p + aq + hd |
| hd | hd | | | | | | |
| KM1465 | KM1466 | KM1467 | KM1468 | KM1469 | KM1470 | KM1471 | KM1472 |
| a + aq + | bz + bc + | bz + cb + hd | bz + cc + hd | bz + lb + hd | bz + lc + hd | bz + sb + hd | bz + sc + |
| hd | hd | | | | | | hd |
| KM1473 | KM1474 | KM1475 | KM1476 | KM1477 | KM1478 | KM1479 | KM1480 |
| bz + aq + hd | bc + cb + | bc + cc + hd | bc + lb + hd | bc + lc + hd | bc + sb + hd | bc + sc + hd | bc + aq + |
| | hd | | | | | | hd |
| KM1481 | KM1482 | KM1483 | KM1484 | KM1485 | KM1486 | KM1487 | KM1488 |
| cb + cc + | cb + lb + | cb + lc + hd | cb + sb + hd | cb + sc + hd | cb + aq + hd | cc + lb + hd | cc + lc + hd |
| hd | hd | | | | | | |
| KM1489 | KM1490 | KM1491 | KM1492 | KM1493 | KM1494 | KM1495 | KM1496 |
| cc + sb + | cc + sc + | cc + aq + hd | lb + lc + hd | lb + sb + hd | lb + sc + hd | lb + aq + hd | lc + sb + hd |
| hd | hd | | | | | | |
| KM1497 | KM1498 | KM1499 | KM1500 | KM1501 | KM1502 | KM1503 | KM1504 |
| lc + sc + | lc + aq + | sb + sc + hd | s + h + b + hd | s + b + u + hd | s + u + p + hd | s + p + pi + | s + pi + e + |
| hd | hd | | | | | hd | hd |
| KM1505 | KM1506 | KM1507 | KM1508 | KM1509 | KM1510 | KM1511 | KM1512 |
| s + e + a + hd | s + a + aq + | s + h + a + | s + h + u + | s + h + hd + | s + h + pi + | s + h + p + | s + b + p + |
| | hd | hd | hd | hd | hd | hd | hd |
| KM1513 | KM1514 | KM1515 | KM1516 | KM1517 | KM1518 | KM1519 | KM1520 |
| s + b + u + | s + h + b + | s + b + a + | s + u + a + | s + b + e + | s + u + e + | s + u + pi + | s + u + p + |
| hd | hd | hd | hd | hd | hd | hd | hd |
| KM1521 | KM1522 | KM1523 | KM1524 | KM1525 | KM1526 | KM1527 | KM1528 |
| s + p + a + hd | s + pi + a + | s + e + a + | s + p + e + | s + pi + e + | s + p + pi + | h + b + a + | h + b + e + |
| | hd | hd | hd | hd | hd | hd | hd |
| KM1529 | KM1530 | KM1531 | KM1532 | KM1533 | KM1534 | KM1535 | KM1536 |
| h + u + e + | h + b + pi + | h + u + pi + | h + b + p + | h + u + p + | h + b + u + | h + u + a + | h + p + a + |
| hd | hd | hd | hd | hd | hd | hd | hd |
| KM1537 | KM1538 | KM1539 | KM1540 | KM1541 | KM1542 | KM1543 | KM1544 |
| h + pi + e + | h + e + a + hd | h + p + e + | h + pi + e + | h + p + pi + | b + u + a + | b + p + a + | b + pi + e + |
| hd | | hd | hd | hd | hd | hd | hd |
| KM1545 | KM1546 | KM1547 | KM1548 | KM1549 | KM1550 | KM1551 | KM1552 |
| b + u + pi + | b + p + pi + | b + u + p + | u + p + a + | u + pi + a + | u + e + a + | u + p + e + | u + pi + e + |
| hd | hd | hd | hd | hd | hd | hd | hd |
| KM1553 | KM1554 | KM1555 | KM1556 | KM1557 | KM1558 | KM1559 | KM1560 |
| u + p + pi + | p + pi + a + | p + pi + e + | pi + e + a + | e + a + aq + | sb + aq + hd | sc + aq + | aq + oc + |
| hd | hd | hd | hd | hd | | hd | hd |
| KM1561 | KM1562 | KM1563 | KM1564 | KM1565 | KM1566 | KM1567 | KM1568 |
| u + hp | p + hp | pi + hp | e + hp | a + hp | bz + hp | bc + hp | cb + hp |
| KM1569 | KM1570 | KM1571 | KM1572 | KM1573 | KM1574 | KM1575 | KM1576 |
| cc + hp | lb + hp | lc + hp | sb + hp | sc + hp | aq + hp | s + h + hp | s + b + hp |
| KM1577 | KM1578 | KM1579 | KM1580 | KM1581 | KM1582 | KM1583 | KM1584 |
| s + u + hp | s + p + hp | s + pi + hp | s + e + hp | s + a + hp | s + bz + hp | s + bc + hp | s + bc + hp |
| KM1585 | KM1586 | KM1587 | KM1588 | KM1589 | KM1590 | KM1591 | KM1592 |
| s + cb + hp | s + cc + hp | s + lb + hp | s + lc + hp | s + sb + hp | s + sc + hp | s + aq + hp | h + b + hp |
| KM1593 | KM1594 | KM1595 | KM1596 | KM1597 | KM1598 | KM1599 | KM1600 |
| h + u + hp | h + p + hp | h + pi + hp | h + e + hp | h + a + hp | h + bz + hp | h + bc + hp | h + bc + hp |
| KM1601 | KM1602 | KM1603 | KM1604 | KM1605 | KM1606 | KM1607 | KM1608 |
| h + cb + | h + cc + hp | h + lb + hp | h + lc + hp | h + sb + hp | h + sc + hp | h + aq + hp | u + p + hp |
| hp | | | | | | | |
| KM1609 | KM1610 | KM1611 | KM1612 | KM1613 | KM1614 | KM1615 | KM1616 |
| u + pi + hp | u + e + hp | u + a + hp | u + bz + hp | u + bc + hp | u + bc + hp | u + cb + hp | u + cc + hp |
| KM1617 | KM1618 | KM1619 | KM1620 | KM1621 | KM1622 | KM1623 | KM1624 |
| u + lb + hp | u + lc + hp | u + sb + hp | u + sc + hp | u + aq + hp | p + pi + hp | p + e + hp | p + a + hp |
| KM1625 | KM1626 | KM1627 | KM1628 | KM1629 | KM1630 | KM1631 | KM1632 |
| p + bz + | p + bc + hp | p + cb + hp | p + cc + hp | p + lb + hp | p + lc + hp | p + sb + hp | p + sc + hp |
| hp | | | | | | | |
| KM1633 | KM1634 | KM1635 | KM1636 | KM1637 | KM1638 | KM1639 | KM1640 |
| pi + e + hp | pi + a + hp | pi + bz + hp | pi + bc + hp | pi + bc + hp | pi + cb + hp | pi + cc + hp | pi + lb + hp |
| KM1641 | KM1642 | KM1643 | KM1644 | KM1645 | KM1646 | KM1647 | KM1648 |
| pi + lc + hp | pi + sb + | pi + sc + hp | pi + aq + hp | e + a + hp | e + bz + hp | e + bc + hp | e + cb + hp |
| | hp | | | | | | |
| KM1649 | KM1650 | KM1651 | KM1652 | KM1653 | KM1654 | KM1655 | KM1656 |
| e + cc + hp | e + lb + hp | e + lc + hp | e + sb + hp | e + sc + hp | e + aq + hp | a + bz + hp | a + bc + hp |
| KM1657 | KM1658 | KM1659 | KM1660 | KM1661 | KM1662 | KM1663 | KM1664 |
| a + bc + | a + cb + | a + cc + hp | a + lb + hp | a + lc + hp | a + sb + hp | a + sc + hp | p + aq + hp |
| hp | hp | | | | | | |
| KM1665 | KM1666 | KM1667 | KM1668 | KM1669 | KM1670 | KM1671 | KM1672 |
| a + aq + | bz + bc + | bz + cb + hp | bz + cc + hp | bz + lb + hp | bz + lc + hp | bz + sb + hp | bz + sc + |
| hp | hp | | | | | | hp |
| KM1673 | KM1674 | KM1675 | KM1676 | KM1677 | KM1678 | KM1679 | KM1680 |
| bz + aq + hp | bc + cb + | bc + cc + hp | bc + lb + hp | bc + lc + hp | bc + sb + hp | bc + sc + hp | bc + aq + |
| | hp | | | | | | hp |
| KM1681 | KM1682 | KM1683 | KM1684 | KM1685 | KM1686 | KM1687 | KM1688 |
| cb + cc + | cb + lb + | cb + lc + hp | cb + sb + hp | cb + sc + hp | cb + aq + hp | cc + lb + hp | cc + lc + hp |
| hp | hp | | | | | | |
| KM1689 | KM1690 | KM1691 | KM1692 | KM1693 | KM1694 | KM1695 | KM1696 |
| cc + sb + | cc + sc + | cc + aq + hp | lb + lc + hp | lb + sb + hp | lb + sc + hp | lb + aq + hp | lc + sb + hp |
| hp | hp | | | | | | |
| KM1697 | KM1698 | KM1699 | KM1700 | KM1701 | KM1702 | KM1703 | KM1704 |
| lc + sc + | lc + aq + | sb + sc + hp | s + h + b + hp | s + b + u + hp | s + u + p + hp | s + p + pi + | s + pi + e + |
| hp | hp | | | | | hp | hp |
| KM1705 | KM1706 | KM1707 | KM1708 | KM1709 | KM1710 | KM1711 | KM1712 |
| s + e + a + hp | s + a + aq + | s + h + a + | s + h + u + | s + h + hp + | s + h + pi + | s + h + p + | s + b + p + |
| | hp | hp | hp | hp | hp | hp | hp |
| KM1713 | KM1714 | KM1715 | KM1716 | KM1717 | KM1718 | KM1719 | KM1720 |
| s + b + u + | s + h + b + | s + b + a + | s + u + a + | s + b + e + | s + u + e + | s + u + pi + | s + u + p + |
| hp | hp | hp | hp | hp | hp | hp | hp |
| KM1721 | KM1722 | KM1723 | KM1724 | KM1725 | KM1726 | KM1727 | KM1728 |
| s + p + a + hp | s + pi + a + | s + e + a + | s + p + e + | s + pi + e + | s + p + pi + | h + b + a + | h + b + e + |
| | hp | hp | hp | hp | hp | hp | hp |
| KM1729 | KM1730 | KM1731 | KM1732 | KM1733 | KM1734 | KM1735 | KM1736 |
| h + u + e + | h + b + pi + | h + u + pi + | h + b + p + | h + u + p + | h + b + u + | h + u + a + | h + p + a + |
| hp | hp | hp | hp | hp | hp | hp | hp |
| KM1737 | KM1738 | KM1739 | KM1740 | KM1741 | KM1742 | KM1743 | KM1744 |
| h + pi + e + | h + e + a + hp | h + p + e + | h + pi + e + | h + p + pi + | b + u + a + | b + p + a + | b + pi + e + |
| hp | | hp | hp | hp | hp | hp | hp |
| KM1745 | KM1746 | KM1747 | KM1748 | KM1749 | KM1750 | KM1751 | KM1752 |
| b + u + pi + | b + p + pi + | b + u + p + | u + p + a + | u + pi + a + | u + e + a + | u + p + e + | u + pi + e + |
| hp | hp | hp | hp | hp | hp | hp | hp |
| KM1753 | KM1754 | KM1755 | KM1756 | KM1757 | KM1758 | KM1759 | KM1760 |
| u + p + pi + | p + pi + a + | p + pi + e + | pi + e + a + | e + a + aq + | sb + aq + hp | sc + aq + | aq + oc + |
| hp | hp | hp | hp | hp | | hp | hp |

| KM1761 | KM1762 |
|---|---|
| h + hp | b + hp |

The previously indicated embodiments of the cosmetic compositions have good deodorant and/or antiperspirant performance and excellent preservation. Because of the synergistic effect of the preservative agents used, the amount thereof can be reduced without negatively affecting the preservative effect. Therefore, the cosmetic agents according to the invention are especially low-irritation and low-sensitization cosmetic agents.

The cosmetic agents according to the invention can also include further auxiliary substances. The antiperspirant cosmetic agents according to the invention preferably include at least one further auxiliary substance, which is selected from the group of (i) emulsifiers and/or surfactants; (ii) hydrogel-forming agents; (iii) chelating agents; (iv) waxes; (v) odorous substances; (vi) skin-cooling active substances; (viii) thickeners; and (ix) mixtures thereof.

Emulsifiers and surfactants that are preferably suitable according to the invention are selected from anionic, cationic, non-ionic, amphoteric, particularly ampholytic and zwitterionic emulsifiers and surfactants. Surfactants are amphiphilic (bifunctional) compounds that consist of at least one hydrophobic molecule part and at least one hydrophilic molecule part. The hydrophobic residue is preferably a hydrocarbon chain having 8 to 28 carbon atoms, which hydrocarbon chain can be saturated or unsaturated, linear or branched. This C₈-C₂₈-alkyl chain is especially preferably linear.

The term “anionic surfactants” is understood to mean surfactants having only anionic charges; they contain, for example, carboxyl groups, sulfonic acid groups, or sulfate groups. Especially preferred anionic surfactants are alkyl sulfates, alkyl ether sulfates, acyl glutamates, and C₈₋₂₄ carboxylic acids and salts thereof, the soaps.

The term “cationic surfactants” is understood to mean surfactants having only cationic charges; they contain, for example, quaternary ammonium groups. Cationic surfactants of the type of the quaternary ammonium compounds, the esterquats, and the amidoamines are preferred. Preferred quaternary ammonium compounds are ammonium halides and the imidazolium compounds known under the INCI names Quaternium-27 and Quaternium-83. Other cationic surfactants that can be used according to the invention are the quaternized protein hydrolysates. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines.

The amphoteric surfactants are divided into ampholytic surfactants and zwitterionic surfactants. The term “ampholytic surfactants” is understood to mean surface-active compounds that have both acidic (for example, —COOH or —SO₃H groups) and basic hydrophilic groups (for example, amino groups) and thus behave acidically or basically depending on the condition. A person skilled in the art understands the term “zwitterionic surfactants” to mean surfactants that bear both a negative charge and a positive charge in the same molecule. Examples of preferred zwitterionic surfactants are the betaines, the N-alkyl-N,N-dimethylammonium glycinates, the N-acyl-aminopropyl-N,N-dimethylammonium glycinates, and the 2-alkyl-3-carboxymethyl-3-hydroxyethyl-imidazolines each having 8 to 24 carbon atoms in the alkyl group. Examples of preferred ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids each having 8 to 24 carbon atoms in the alkyl group.

The compositions according to the invention that are formulated as an emulsion, particularly as an oil-in-water emulsion, preferably include at least one non-ionic oil-in-water emulsifier having an HLB of more than 7 to 20. These are emulsifiers well known to a person skilled in the art, which are listed, for example, in Kirk-Othmer, “Encyclopedia of Chemical Technology”, 3rd edition, 1979, volume 8, pages 913-916. For ethoxylated products, the HLB is calculated according to the formula HLB=(100−L):5, wherein L is the proportion by weight of the lipophilic groups, i.e., the fatty alkyl or fatty acyl groups, in the ethylene oxide adducts, expressed in weight percent. In this context, it can be preferred according to the invention if, furthermore, a water-in-oil emulsifier having an HLB of greater than 1.0 and less than or equal to 7.0 is used. Non-ionic oil-in-water emulsifiers and non-ionic water-in-oil emulsifiers that are suitable in the context of the present invention are described, for example, in German laid-open application DE 102006004957 A1. Especially well-suited non-ionic surfactants are, for example, silicone copolyols having ethylene oxide units or having ethylene oxide units and propylene oxide units, which likewise are disclosed in laid-open application DE 102006004957 A1.

To thicken the antiperspirant cosmetic agents according to the invention, preferably hydrogel-forming substances are used, which are selected from cellulose ethers, especially hydroxyalkyl celluloses, particularly hydroxypropyl cellulose, hydroxypropyl methylcellulose, hydroxyethyl cellulose, carboxymethyl cellulose, cetyl hydroxyethylcellulose, hydroxybutyl methylcellulose, methylhydroxyethyl cellulose; and xanthan gum, sclerotium gum, succinoglucans, polygalactomannans, particularly guar gums and locust bean gum, particularly guar gum and locust bean gum themselves and the non-ionic hydroxyalkyl guar derivatives and locust bean gum derivatives, such as hydroxypropyl guar, carboxymethyl hydroxypropyl guar, hydroxypropyl methyl guar, hydroxyethyl guar, and carboxymethyl guar; and pectins, agar, carrageenan, tragacanth, gum arabic, gum karaya, tara gum, gellan gum, gelatin, casein, propylene glycol alginate, alginic acids and salts thereof, particularly sodium alginate, potassium alginate, and calcium alginate; and polyvinylpyrrolidones, polyvinyl alcohols, polacrylamides; and—even if less preferably—starches that are physically modified (e.g., by means of pre-gelatinization) and/or chemically modified, particularly hydroxypropylated starch phosphates and octenyl starch succinates and aluminum, calcium, or sodium salts thereof; and—likewise less preferably—acrylic acid/acrylate copolymers, acrylic acid/acrylamide copolymers, acrylic acid/vinylpyrrolidone copolymers, acrylic acid/vinylformamide copolymers, and polyacrylates. Especially preferred hydrogel-forming agents are selected from cellulose ethers, especially from hydroxyalkyl celluloses, particularly from hydroxypropyl cellulose, hydroxypropyl methylcellulose, hydroxyethyl cellulose, carboxymethyl cellulose, cetyl hydroxyethylcellulose, hydroxybutyl methylcellulose, and methylhydroxyethyl cellulose, and mixtures thereof. Hydroxyethyl cellulose is preferably used as a hydrogel-forming agent.

Furthermore, the cosmetic agents can include at least one chelating agent, which is preferably selected from ethylenediamine tetraacetic acid (EDTA) and salts thereof and from nitrilotriacetic acid (NTA) and mixtures of these substances, in a total amount of 0.01 to 0.5 wt %, preferably 0.02 to 0.3 wt %, particularly 0.05 to 0.1 wt %, with respect to the total weight of the propellant-free cosmetic agent.

In addition, the cosmetic agents according to the invention can include an odorous substance. In the sense of the present invention, the term “odorous substances” is understood to mean substances having a molar mass of 74 to 300 g/mol that include at least one osmophore in the molecule and have an odor and/or flavor, i.e., they are capable of exciting the receptors of the hair cells of the olfactory system. Osmophores are groups covalently bonded to the molecular skeleton, in the form of hydroxy groups, formyl groups, oxo groups, alkoxycarbonyl groups, nitrile groups, nitro groups, azide groups, etc. In this context, perfume oils, perfumes, or perfume oil constituents that are liquid at 20° C. and 1,013 hPa also fall under the term “odorous substances” in the sense of the present invention. The odorous substance is preferably selected from the group of

(i) esters, particularly benzyl acetate, phenoxyethyl isobutyrate, p-tert-butylcyclohexyl acetate, linalyl acetate, dimethyl benzyl carbinyl acetate (DMBCA), phenethyl acetate, benzyl acetate, ethyl methyl phenyl glycinate, allyl cyclohexyl propionate, styrallyl propionate, benzyl salicylate, cyclohexyl salicylate, Floramat, Melusat, and Jasmecyclat;
(ii) ethers, particularly benzyl ethyl ether and Ambroxan;
(iii) aldehydes, particularly linear alkanals having 8 to 18 carbon atoms, citral, citronellal, citronellyloxy acetaldehyde, cyclamen aldehyde, Lilial, and bourgeonal;
(iv) ketones, particularly ionones, alpha-isomethyl ionone, and methyl cedryl ketone;
(v) alcohols, particularly anethole, citronellol, eugenol, geraniol, linalool, phenethyl alcohol, and terpineol;
(vi) hydrocarbons, particularly terpenes such as limonene and pinene; and
(vii) mixtures thereof.
Mixtures of different odorous substances which jointly produce a pleasant fragrance are preferably used.

Especially pleasant-smelling cosmetic agents according to the invention are obtained if the odorous substance is contained in a total amount of 0.00001 to 10 wt %, preferably 0.001 to 9 wt %, preferably 0.01 to 8 wt %, more preferably 0.5 to 7 wt %, particularly 1 to 6 wt %, with respect to the total weight of the propellant-free cosmetic agent.

In addition, the cosmetic agents according to the invention can include at least one wax. In the context of the present invention, the term “waxes” should be understood to mean substances that are kneadable or solid to brittle and hard at 20° C., have a coarse to finely crystalline structure, and are colorfully translucent to opaque, but not vitreous. Furthermore, these substances melt above 25° C. without decomposition, flow readily (have low viscosity) slightly above the melting point, have a highly temperature-dependent consistency and solubility, and can be polished under light pressure. The wax can preferably be selected from the group of

(i) coconut fatty acid glycerol mono-, di-, and triesters;

### (ii) Butyrospermum Parkii (Shea Butter);

(iii) esters of saturated, monohydric C₈₋₁₈ alcohols with saturated C₁₂₋₁₈ monocarboxylic acids;
(iv) linear, primary C₁₂-C₂₄ alkanols;
(v) esters of a saturated, monohydric C₁₆-C₆₀ alkanol and a saturated C₈-C₃₆ monocarboxylic acid, particularly cetyl behenate, stearyl behenate, and C₂₀-C₄₀ alkyl stearate;
(vi) glycerol triesters of saturated linear C₁₂-C₃₀ carboxylic acids, which can be hydroxylated, particularly hydrogenated palm oil, hydrogenated coconut oil, hydrogenated castor oil, glyceryl tribehenate, and glyceryl tri(12-hydroxystearate);
(vii) natural plant waxes, particularly candelilla wax, carnauba wax, Japan wax, sugarcane wax, ouricury wax, cork wax, sunflower wax, fruit waxes;
(viii) animal waxes, particularly beeswax, shellac wax, and spermaceti;
(ix) synthetic waxes, particularly montan ester waxes, hydrogenated jojoba waxes and Sasol waxes, polyalkylene waxes and polyethylene glycol waxes, C₂₀-C₄₀ dialkyl esters of dimer acids, C₃₀₋₅₀ alkyl beeswax, and alkyl and alkyl aryl esters of dimer fatty acids, paraffin waxes; and
(x) mixtures thereof

Especially preferred are commercial products having the INCI name Cocoglycerides, particularly the commercial products Novata® (from BASF), especially preferably Novata® AB, a mixture of C₁₂₋₁₈ mono-, di-, and triglycerides that melts in the range of 30 to 32° C., and the products of the Softisan series (Sasol Germany GmbH) having the INCI name Hydrogenated Cocoglycerides, particularly Softisan 100, 133, 134, 138, 142. Other preferred esters of saturated, monohydric C₁₂₋₁₈ alcohols with saturated C₁₂₋₁₈ monocarboxylic acids are stearyl laurate, cetearyl stearate (e.g., Crodamol® CSS), cetyl palmitate (e.g., Cutina® CP), and myristyl myristate (e.g., Cetiol® MM). Furthermore, a C₂₀-C₄₀ alkyl stearate is preferably used as a wax component. This ester is known under the names Kester Wax® K82H and Kester Wax® K80H and is sold by Koster Keunen Inc.

In the context of the present invention, it is preferred if the wax is contained in a total amount of 0.01 to 20 wt %, preferably 3 to 20 wt %, preferably 5 to 18 wt %, particularly 6 to 15 wt %, with respect to the total weight of the propellant-free cosmetic agent.

According to another embodiment of the present invention, the antiperspirant cosmetic agents also include at least one skin-cooling active substance. Skin-cooling active substances that are suitable according to the invention are, for example, menthol, isopulegol, and menthol derivatives, e.g., menthyl lactate, menthyl glycolate, menthyl ethyl oxamate, menthyl pyrrolidone carboxylic acid, menthyl methyl ether, menthoxypropanediol, menthone glycerin acetal (9-methyl-6-(1-methylethyl)-1,4-dioxaspiro[4.5]decane-2-methanol), monomenthyl succinate, 2-hydroxymethyl-3,5,5-trimethylcyclohexanol, and 5-methyl-2-(1-methylethyl)cyclohexyl-N-ethyloxamate. Menthol, isopulegol, menthyl lactate, menthoxypropanediol, menthyl pyrrolidone carboxylic acid, and 5-methyl-2-(1-methylethyl)cyclohexyl-N-ethyloxamate and mixtures of these substances, particularly mixtures of menthol and menthyl lactate, menthol, menthol glycolate, and menthyl lactate, menthol and menthoxypropanediol, or menthol and isopulegol, are preferred as skin-cooling active substances.

Furthermore, according to the invention, lipophilic thickeners can be used as auxiliary substances. The at least one antiperspirant aluminum salt is preferably suspended undissolved in at least one cosmetic oil that is liquid at 20° C. and 1,013 hPa. To improve usability, at least one lipophilic thickener can be added to this suspension as a suspension aid. Lipophilic thickeners preferred according to the invention are selected from hydrophobed clay minerals and pyrogenic silicic acids.

In addition, all other components known to a person skilled in the art for such cosmetic compositions can, in principle, be used the cosmetic agents according to the invention. Further active substances, auxiliary substances, and additives are, for example:
structurants such as maleic acid and lactic acid,
fiber-structure-improving active substances, particularly mono-, di-, and oligosaccharides, such as glucose, galactose, fructose, fruit sugar, and lactose,
dyes for dyeing the agent,
substances for setting the pH value, such as α- and β-hydroxycarboxylic acids,
active substances such as allantoin and bisabolol,
ceramides. The term “ceramides” is understood to mean N-acylsphingosine (fatty acid amides of sphingosine) or synthetic analogs of such lipids (pseudo-ceramides),
pigments,
viscosity regulators such as salts (NaCl),
cationic, non-ionic, and amphoteric polymers,
vitamins, particularly from the groups A, B, C, E, F, and H,
protein hydrolysates and cationized protein hydrolysates,
humectants or penetration enhancers and/or swelling agents, particularly urea and urea derivatives, guanidine and derivatives thereof, arginine and derivatives thereof, water glass, imidazole and derivatives thereof, histidine and derivatives thereof,
plant extracts, for example from green tea, white tea, oak bark, nettles, witch hazel, hops, chamomile, burdock root, horsetail, whitethorn, linden blossoms, lychee, almond, aloe vera, spruce needle, Aesculus, sandalwood, juniper, coconut, mango, apricot, lime, wheat, kiwi, melon, orange, grapefruit, sage, rosemary, birch, mallow, meadowfoam, wild thyme, yarrow, garden thyme, melissa, restharrow, coltsfoot, marshmallow, ginseng, ginger root, Echinacea purpurea, Olea europea, Foeniculum vulgaris, and Apim graveolens.

The previously mentioned further ingredients can be contained in a total amount of 0.001 to 50 wt %, preferably 0.01 to 40 wt %, preferably 0.1 to 30 wt %, particularly 0.5 to 20 wt %, with respect to the total weight of the propellant-free cosmetic agent.

The present invention also relates to a non-therapeutic cosmetic method for preventing and/or reducing the perspiration of the body and/or for preventing and/or reducing body odor, wherein a cosmetic agent according to the invention is applied to the skin, particularly to the skin of the armpits, and remains on the skin for at least 1 hour, preferably for at least 2 hours, preferably for at least 4 hours, particularly for at least 6 hours.

That which was said with regard to the cosmetic agents according to the invention applies, mutatis mutandis, to other preferred embodiments of the method according to the invention, particularly to the cosmetic agents used there.

Finally, the present invention also relates to the use of at least two preservatives, selected from the group of sulfite(s), hexetidine, benzyl alcohol(s), undecylenic acid, phenoxyisopropanol, piroctone olamine, ethyl lauroyl arginate, formic acid, quaternary ammonium compounds, and o-cymen-5-ol, to preserve propellant-containing deodorant and/or antiperspirant compositions.

The use of the previously mentioned preservative-agent combinations leads to excellent preservation of propellant-containing deodorant and/or antiperspirant compositions. Because of the synergistic effect of said combinations with respect to the preservative effect, the amount of preservative agents can be reduced so that low-irritation and low-sensitization deodorant and/or antiperspirant compositions result. Here, the term “propellant-containing deodorant and antiperspirant compositions” is understood to mean compositions that include at least one propellant and at least one deodorant active substance and/or at least one antiperspirant active substance.

That which was said with regard to the cosmetic agents according to the invention and the method according to the invention applies, mutatis mutandis, to other embodiments of the use according to the invention.

In summary, the present invention is characterized in particular by the following points:
1. A cosmetic agent containing, in a cosmetically acceptable carrier,
   a) 0.0005 to 35 wt %, with respect to the total weight of the propellant-free cosmetic agent, of at least one active substance, selected from the group of deodorant active substances, antiperspirant aluminum salts, and mixtures thereof,
   b) 2.0 to 95 wt %, with respect to the total weight of the propellant-containing cosmetic agent, of at least one propellant, and
   c) at least two preservative agents, selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.
2. The cosmetic agent according to point 1, characterized in that said cosmetic agent includes at least one deodorant active substance, selected from the group of (i) silver salts; (ii) alkane diols having 5 to 12 carbon atoms, particularly 3-(2-ethylhexyloxy)-1,2-propanediol, 1,2-hexanediol, and 1,2-octanediol; (iii) triethyl citrates; (iv) active substances against exo-esterases, particularly against aryl sulfatase, lipase, beta-glucuronidase, and cystathionine β-lyase; (v) cationic phospholipids; (vi) odor absorbers, particularly silicates, such as montmorillonite, kaolinite, illite, beidellite, nontronite, saponite, hectorite, bentonite, smectite and talc, zeolites, zinc ricinoleate, cyclodextrins; (vii) deodorizing ion exchangers; (viii) microbe-inhibiting agents; (ix) prebiotic components; and (x) mixtures thereof
3. The cosmetic agent according to one of points 1 and 2, characterized in that said cosmetic agent includes—with respect to the total weight of the propellant-free cosmetic agent—0.0005 to 20 wt %, preferably 0.1 to 12 wt %, preferably 0.1 to 10 wt %, particularly 0.1 to 2.0 wt %, of at least one deodorant active substance. 4. The cosmetic agent according to one of the preceding points, characterized in that said cosmetic agent includes at least one antiperspirant aluminum salt, selected from the group of (i) water-soluble astringent inorganic salts of aluminum, particularly aluminum chlorohydrate, aluminum sesquichlorohydrate, aluminum dichlorohydrate, aluminum hydroxide, potassium aluminum sulfate, aluminum bromohydrate, aluminum chloride, aluminum sulfate; (ii) water-soluble astringent organic salts of aluminum, particularly aluminum chlorohydrex propylene glycol, aluminum chlorohydrex polyethylene glycol, aluminum propylene glycol complexes, aluminum sesquichlorohydrex propylene glycol, aluminum sesquichlorohydrex polyethylene glycol, aluminum dichlorohydrex propylene glycol, aluminum dichlorohydrex polyethylene glycol, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate; and (iii) mixtures thereof.
5. The cosmetic agent according to one of the preceding points, characterized in that said cosmetic agent includes—with respect to the total weight of the propellant-free cosmetic agent—0.1 to 35 wt %, preferably 0.5 to 25 wt %, preferably 1 to 15 wt %, more preferably 1.5 to 10 wt %, particularly 2.0 to 8.0 wt %, of at least one antiperspirant aluminum salt.
6. The cosmetic agent according to one of the preceding points, characterized in that said cosmetic agent includes at least one propellant, selected from the group of propane, propene, n-butane, isobutane, isobutylene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, carbon dioxide, and mixtures thereof
7. The cosmetic agent according to one of the preceding points, characterized in that said cosmetic agent includes—with respect to the total weight of the propellant-free cosmetic agent—2.0 to 90 wt %, preferably 30 to 85 wt %, preferably 35 to 80 wt %, particularly 40 to 75 wt %, of at least one propellant.
8. The cosmetic agent according to one of the preceding points, characterized in that said cosmetic agent includes at least three preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.
9. The cosmetic agent according to one of the preceding points, characterized in that said cosmetic agent includes at least four preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.
10. The cosmetic agent according to one of the preceding points, characterized in that said cosmetic agent includes at least five preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.
11. The cosmetic agent according to one of the preceding points, characterized in that said cosmetic agent includes at least six preservative agents (c), selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.
12. The cosmetic agent according to one of the preceding points, characterized in that said cosmetic agent includes a mixture of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine as a preservative agent (c).
13. The cosmetic agent according to one of the preceding points, characterized in that said cosmetic agent includes at least one compound, selected from the group of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and mixtures thereof, as quaternary ammonium compounds.
14. The cosmetic agent according to one of the preceding points, characterized in that said cosmetic agent includes—with respect to the total weight of the propellant-free cosmetic agent—0.001 to 10 wt %, preferably 0.005 to 7.0 wt %, preferably 0.01 to 4.0 wt %, particularly 0.05 to 2.0 wt %, of at least two preservative agents (c).
15. A non-therapeutic cosmetic method for preventing and/or reducing the perspiration of the body and/or for preventing and/or reducing body odor, wherein a cosmetic agent according to one of points 1 to 14 is applied to the skin, particularly to the skin of the armpits, and remains on the skin for at least 1 hour, preferably for at least 2 hours, preferably for at least 4 hours, particularly for at least 6 hours.
16. The use of at least two preservative agents, selected from the group of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine, to preserve propellant-containing deodorant and/or antiperspirant compositions.

The following examples illustrate the present invention without restricting the present invention thereto:

### Examples

Sprayable, translucent antiperspirant microemulsions (specifications in wt %, with respect to the microemulsion without propellant)

| | 1.1 | 1.2 | 1.3 | 1.4 | 1.5 | 1.6 |
|---|---|---|---|---|---|---|
| Plantaren ® 1200 | 1.7 | 1.7 | — | 1.7 | 1.7 | — |
| Plantaren ® 2000 | 1.15 | 1.4 | 2.40 | 1.15 | 1.4 | 2.4 |
| Glycerol monooleate | 0.7 | 0.7 | — | 0.7 | 0.7 | — |
| Dioctyl ether | 4 | 4. | 0.1 | 4 | 4 | 0.1 |
| Octyldodecanol | 1 | 1 | 0.02 | 1 | 1 | 0.02 |
| Perfume oil | 1 | 1 | 1 | 1 | 1 | 10 |
| Aluminum chlorohydrate | 8 | 5 | 5 | 10 | 9 | 13 |
| 1,2-Propylene glycol | 5 | 5 | — | 5 | 5 | 5 |
| Glycerol | — | — | 5 | — | — | — |
| 2-Benzylheptan-1-ol | 0.5 | — | — | 0.5 | 0.5 | — |
| Triethyl citrate | — | 0.5 | 0.5 | 0.2 | — | — |
| Triclosan | — | — | — | — | — | 0.5 |
| Zinc lactate | — | 0.2 | — | — | — | — |
| Sodium sulfite | 0.2 | — | — | — | — | 0.2 |
| Hexetidine | 0.1 | 0.1 | 0.1 | — | — | 0.1 |
| Benzyl alcohol | — | 1.0 | — | — | — | 1.0 |
| Undecylenic acid | — | — | 0.2 | — | — | 0.2 |
| Phenoxyisopropanol | — | — | — | — | — | 1.0 |
| Piroctone olamine | — | — | — | — | — | 1.0 |
| Ethyl lauroyl arginate HCl | — | — | — | — | — | 0.4 |
| Formic acid | — | — | — | — | — | 0.5 |
| o-Cymen-5-ol | — | — | — | — | — | 0.1 |
| Behentrimonium chloride | — | — | — | — | — | 0.1 |
| Cetrimonium bromide | — | — | — | — | — | 0.1 |
| Cetrimonium chloride | — | — | — | — | — | 0.1 |
| Laurtrimonium bromide | — | — | — | — | — | 0.1 |
| Laurtrimonium chloride | — | — | — | — | — | 0.1 |
| Steartrimonium bromide | — | — | — | — | — | 0.1 |
| Steartrimonium chloride | — | — | — | — | — | 0.1 |
| Benzethonium chloride | — | — | — | — | — | 0.1 |
| Benzalkonium chloride | — | — | — | — | — | 0.1 |
| Hexamidine | — | — | — | — | — | 0.1 |
| Hexamidine diisethionate | — | — | — | — | — | 0.1 |
| Hexamidine paraben | — | — | — | — | — | 0.1 |
| KM¹⁾ | — | — | — | 0.1 | 2.0 | — |
| Water | Ad 100 | Ad 100 | Ad 100 | Ad 100 | Ad 100 | Ad 100 |

¹⁾Preservative-agent mixture, selected from at least one of the previously mentioned preservative-agent mixtures KM1 to KM1762

Microemulsions 1.1 to 1.6 are applied from a pressure vessel (aerosol) by means of a suitable spraying system. Air, nitrous oxide, nitrogen, CO₂, and other compressible gases are used as propellants.

Antiperspirant emulsion (O/W) (specifications in wt %, with respect to the O/W emulsion without propellant)

| | 2.1 | 2.2 | 2.3 | 2.4 |
|---|---|---|---|---|
| Aluminum Zirconium | 23.7 | — | 23.7 | — |
| Tetrachlorohydrex Gly | | | | |
| Locron L | — | 30.0 | — | 30.0 |
| Steareth-2 | 2.4 | 2.2 | 2.4 | 2.2 |
| Steareth-21 | 1.6 | 1.4 | 1.6 | 1.4 |
| Perfume | 1.2 | 1.0 | 1.2 | 1.0 |
| PPG-15 Stearyl Ether | 0.5 | 1.5 | 0.5 | 1.5 |
| Aluminum Starch | 0.1 | — | 0.1 | — |
| Octenylsuccinate | | | | |
| Sodium sulfite | — | — | — | 0.2 |
| Hexetidine | 0.1 | 0.1 | — | 0.1 |
| Benzyl alcohol | — | — | — | 1.0 |
| Undecylenic acid | — | — | — | 0.2 |
| Phenoxyisopropanol | 1.0 | — | — | 1.0 |
| Piroctone olamine | — | 1.0 | — | 1.0 |
| Ethyl lauroyl arginate HCl | — | — | — | 0.4 |
| Formic acid | — | — | — | 0.5 |
| o-Cymen-5-ol | — | — | — | 0.1 |
| Behentrimonium chloride | — | — | — | 0.1 |
| Cetrimonium bromide | — | — | — | 0.1 |
| Cetrimonium chloride | — | — | — | 0.1 |
| Laurtrimonium bromide | — | — | — | 0.1 |
| Laurtrimonium chloride | — | — | — | 0.1 |
| Steartrimonium bromide | — | — | — | 0.1 |
| Steartrimonium chloride | — | — | — | 0.1 |
| Benzethonium chloride | — | — | — | 0.1 |
| Benzalkonium chloride | — | — | — | 0.1 |
| Hexamidine | — | — | — | 0.1 |
| Hexamidine diisethionate | — | — | — | 0.1 |
| Hexamidine paraben | — | — | — | 0.1 |
| KM¹⁾ | — | — | 4.0 | — |
| Aqua | Ad 100.0 | Ad 100.0 | Ad 100.0 | Ad 100.0 |

¹⁾Preservative-agent mixture, selected from at least one of the previously mentioned preservative-agent mixtures KM1 to KM1762

Emulsions 2.1 to 2.5 are applied from a pressure vessel (aerosol) by means of a suitable spraying system. Air, nitrous oxide, nitrogen, CO₂, and other compressible gases are used as propellants.

Deodorant (specifications in wt %, with respect to the deodorant without propellant)

| | 3.1 | 3.2 | 3.3 | 3.4 |
|---|---|---|---|---|
| 96% ethanol (cosmetic; denatured) | 55.0 | 55.0 | 55.0 | 55.0 |
| Triethyl citrate | 3.0 | 3.0 | 3.0 | 3.0 |
| Cremophor ® RH 455 | 0.5 | 0.5 | 0.5 | 0.5 |
| Eucarol ® AGE-EC-UP | 1.0 | 1.0 | 1.0 | 1.0 |
| Cocamidopropyl PG-Dimonium | 0.4 | 0.4 | 0.4 | 0.4 |
| Chloride Phosphate | | | | |
| Perfume oil | 1.0 | 1.0 | 1.0 | 1.0 |
| Sodium sulfite | — | — | — | 0.2 |
| Hexetidine | 0.1 | — | — | 0.1 |
| Benzyl alcohol | — | — | — | 1.0 |
| Undecylenic acid | — | 0.2 | — | 0.2 |
| Phenoxyisopropanol | — | — | — | 1.0 |
| Piroctone olamine | — | — | — | 1.0 |
| Ethyl lauroyl arginate HCl | 0.4 | — | — | 0.4 |
| Formic acid | — | 0.5 | — | 0.5 |
| o-Cymen-5-ol | — | — | — | 0.1 |
| Behentrimonium chloride | — | — | — | 0.1 |
| Cetrimonium bromide | — | — | — | 0.1 |
| Cetrimonium chloride | — | — | — | 0.1 |
| Laurtrimonium bromide | — | — | — | 0.1 |
| Laurtrimonium chloride | — | — | — | 0.1 |
| Steartrimonium bromide | — | — | — | 0.1 |
| Steartrimonium chloride | — | — | — | 0.1 |
| Benzethonium chloride | — | — | — | 0.1 |
| Benzalkonium chloride | — | — | — | 0.1 |
| Hexamidine | — | — | — | 0.1 |
| Hexamidine diisethionate | — | — | — | 0.1 |
| Hexamidine paraben | — | — | — | 0.1 |
| KM¹⁾ | — | — | 0.2 | — |
| Water | Ad | Ad | Ad | Ad |
| | 100.0 | 100.0 | 100.0 | 100.0 |

¹⁾Preservative-agent mixture, selected from at least one of the previously mentioned preservative-agent mixtures KM1 to KM1762

Deodorant 3.1 to 3.5 is applied from a pressure vessel (aerosol) by means of a suitable spraying system. Air, nitrous oxide, nitrogen, CO₂, and other compressible gases are used as propellants.

Water-in-oil emulsions according to the invention (all amount specifications in wt %, with respect to the total weight of the water-in-oil emulsion without propellant)

| | 4.1 | 4.2 | 4.3 | 4.4 | 4.5 |
|---|---|---|---|---|---|
| Aluminum chlorohydrate | 33 | 33 | 33 | 33 | 33 |
| C₁₀-C₁₃ isoalkane | 8.9 | 8.9 | — | 8.9 | — |
| Cyclopentasiloxane | — | — | 8.9 | — | 8.9 |
| PEG/PPG-18/18 | 1.4 | 1.4 | 1.4 | 1.4 | 1.4 |
| Dimethicone | | | | | |
| Isoceteth-20 | 0.5 | 0.5 | 0.5 | 0.5 | 0.5 |
| Dimethicone | 4.2 | 4.2 | 4.2 | 4.2 | 4.2 |
| Isopropyl myristate | 9.0 | 9.0 | 9.0 | 9.0 | 9.0 |
| 1,2-Propanediol | 7.0 | 25 | 25 | 7.0 | 25 |
| Phenoxyethanol | 0.5 | 0.5 | 0.5 | 0.5 | 0.5 |
| Perfume | 2.5 | 2.5 | 2.5 | 2.5 | 2.5 |
| L-Menthol | 0.4 | — | — | 0.4 | — |
| Sodium sulfite | 0.2 | 0.2 | — | — | 0.2 |
| Hexetidine | — | — | — | — | 0.1 |
| Benzyl alcohol | 1.0 | — | 0.8 | — | 1.0 |
| Undecylenic acid | — | 0.2 | — | — | 0.2 |
| Phenoxyisopropanol | — | — | — | — | 1.0 |
| Piroctone olamine | — | — | 0.8 | — | 1.0 |
| Ethyl lauroyl arginate | — | — | — | — | 0.4 |
| HCl | | | | | |
| Formic acid | — | — | — | — | 0.5 |
| o-Cymen-5-ol | — | — | 0.1 | — | 0.1 |
| Behentrimonium | — | — | — | — | 0.1 |
| chloride | | | | | |
| Cetrimonium bromide | — | — | — | — | 0.1 |
| Cetrimonium chloride | — | — | — | — | 0.1 |
| Laurtrimonium bromide | — | — | — | — | 0.1 |
| Laurtrimonium chloride | — | — | — | — | 0.1 |
| Steartrimonium bromide | — | — | — | — | 0.1 |
| Steartrimonium chloride | — | — | — | — | 0.1 |
| Benzethonium chloride | — | — | — | — | 0.1 |
| Benzalkonium chloride | — | — | — | — | 0.1 |
| Hexamidine | — | — | 0.1 | — | 0.1 |
| Hexamidine | — | — | 0.1 | — | 0.1 |
| diisethionate | | | | | |
| Hexamidine paraben | — | — | 0.1 | — | 0.1 |
| KM¹⁾ | — | — | — | 1.0 | — |
| Water | Ad 100 | Ad 100 | Ad 100 | Ad 100 | Ad 100 |

¹⁾Preservative-agent mixture, selected from at least one of the previously mentioned preservative-agent mixtures KM1 to KM1762

Emulsion 4.1 to 4.5 is applied from a pressure vessel (aerosol) by means of a suitable spraying system. 15, 20, 25, or 30% of the formulation is introduced, together with a suitable propellant gas such as a propane/butane/isobutane mixture (pressure: 1.6 to 3.8 bar), into the aerosol container (ad 100).

Emulsion according to the PIT method (all specifications in wt %, with respect to the propellant-free emulsion):

| | 5.1 | 5.2 | 5.3 | 5.4 |
|---|---|---|---|---|
| Emulgade ® SE-PF | 5 | 35.6 | 5 | 35.6 |
| Eumulgin ® B1 | 3 | 13 | 3 | 13 |
| Cetiol ® SN | 5 | 6 | 5 | 6 |
| Cetiol ® CC | 5 | 8 | 5 | 8 |
| 86% glycerol | 0.1 | 0.2 | 0.1 | 0.2 |
| Perfume | 1 | 1 | 1 | 1 |
| Sodium sulfite | — | — | — | 0.2 |
| Hexetidine | 0.1 | — | — | 0.1 |
| Benzyl alcohol | — | 0.8 | — | 1.0 |
| Undecylenic acid | — | — | — | 0.2 |
| Phenoxyisopropanol | — | — | — | 1.0 |
| Piroctone olamine | — | — | — | 1.0 |
| Ethyl lauroyl arginate HCl | — | 0.3 | — | 0.4 |
| Formic acid | — | — | — | 0.5 |
| o-Cymen-5-ol | 0.1 | — | — | 0.1 |
| Behentrimonium chloride | — | — | — | 0.1 |
| Cetrimonium bromide | — | — | — | 0.1 |
| Cetrimonium chloride | — | 0.1 | — | 0.1 |
| Laurtrimonium bromide | — | — | — | 0.1 |
| Laurtrimonium chloride | — | — | — | 0.1 |
| Steartrimonium bromide | 0.1 | — | — | 0.1 |
| Steartrimonium chloride | — | — | — | 0.1 |
| Benzethonium chloride | — | — | — | 0.1 |
| Benzalkonium chloride | — | — | — | 0.1 |
| Hexamidine | 0.1 | — | — | 0.1 |
| Hexamidine diisethionate | — | 0.1 | — | 0.1 |
| Hexamidine paraben | — | — | — | 0.1 |
| KM¹⁾ | — | — | 0.8 | — |
| Water | Ad 100 | Ad 100 | Ad 100 | Ad 100 |

¹⁾ Preservative-agent mixture, selected from at least one of the previously mentioned preservative-agent mixtures KM1 to KM1762

Emulsion 5.1 to 5.5 is applied from a pressure vessel (aerosol) by means of a suitable spraying system. Air, nitrous oxide, nitrogen, CO₂, and other compressible gases are used as propellants.

The following commercial products were used:

| Commercial | | Supplier/ |
|---|---|---|
| product | INCI | manufacturer |
| Cetiol ® SN | CETEARYL ISONONANOATE | BASF |
| Cetiol ® CC | DICAPRYLYL CARBONATE | BASF |
| Cremophor ® | PEG-60 HYDROGENATED CASTOR | BASF |
| RH 455 | OIL | |
| Eucarol ® | SODIUM COCO-GLUCOSIDE | Cesalpinia |
| AGE-EC-UP | TARTRATE | |
| Dow Corning | Dimethicone, PEG/PPG-18/18 | Dow |
| ES-5227 DM | Dimethicone in a weight ratio of 3:1 | Corning |
| Emulgade SE-PF | GLYCERYL STEARATE, | BASF |
| | CETEARETH-20, CETEARETH-12, | |
| | CETEARYL ALCOHOL, CETYL | |
| | PALMITATE | |
| Eumulgin ® B1 | Ceteareth-12 | BASF |
| Finsolv TN | C12-15 Alkyl Benzoate | Innospec |
| Locron L (AS = | Aluminum Chlorohydrate | Clariant |
| 50%) | | |
| Plantaren ® 1200 | LAURYL GLUCOSIDE, approximately | BASF |
| | 50% AS | |
| Plantaren ® 2000 | DECYL GLUCOSIDE, approximately | BASF |
| | 50% AS | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent containing, in a cosmetically acceptable carrier,
a) 0.0005 to 35 wt %, with respect to the total weight of the propellant-free cosmetic agent, of at least one active substance, selected from the group of deodorant active substances, antiperspirant aluminum salts, and mixtures thereof,
b) 2.0 to 95 wt %, with respect to the total weight of the propellant-containing cosmetic agent, of at least one propellant, and
c) at least two preservative agents, selected from the group consisting of: sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

2. The cosmetic agent according to claim 1, wherein the at least one doodorant active substance comprises, with respect to the total weight of the propellant-free cosmetic agent, 0.0005 to 20 wt %.

3. The cosmetic agent according to claim 1, wherein the at least one doodorant active substance comprises, with respect to the total weight of the propellant-free cosmetic agent, 0.1 to 12 wt %

4. The cosmetic agent according to claim 1, wherein the at least one doodorant active substance comprises, with respect to the total weight of the propellant-free cosmetic agent, 0.1 to 10 wt %.

5. The cosmetic agent according to claim 1, wherein the at least one doodorant active substance comprises, with respect to the total weight of the propellant-free cosmetic agent, 0.1 to 2.0 wt %.

6. The cosmetic agent according to one claim 1, wherein the at least antiperspirant is at least one antiperspirant aluminum salt and comprises 0.1 to 35 wt % of the total weight of the propellant-free cosmetic agent.

7. The cosmetic agent according to claim 1, wherein the cosmetic agent includes at least four preservative agents (c) selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

8. The cosmetic agent according to claim 1, wherein the cosmetic agent includes at least least five preservative agents (c), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine.

9. The cosmetic agent according to claim 1, wherein the cosmetic agent includes a mixture of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, hexamidine, and salts of hexamidine as a preservative agent (c).

10. The cosmetic agent according to claim 1, wherein at least two preservative agents (c) comprise 0.005 to 7.0 wt %, with respect to the total weight of the propellant-free cosmetic agent.

11. The cosmetic agent according to claim 1, wherein at least two preservative agents (c) comprise 0.001 to 10 wt %, with respect to the total weight of the propellant-free cosmetic agent.

12. The cosmetic agent according to claim 1, wherein at least two preservative agents (c) comprise 0.01 to 4.0 wt %, with respect to the total weight of the propellant-free cosmetic agent.

13. The cosmetic agent according to claim 1, wherein at least two preservative agents (c) comprise 0.05 to 2.0 wt %, with respect to the total weight of the propellant-free cosmetic agent.

14. A non-therapeutic cosmetic method for preventing and/or reducing the perspiration of the body and/or for preventing and/or reducing body odor, wherein a cosmetic agent according to claim 1 is applied to the skin, and is left the skin for at least 1 hour.
